(19) United States
(12) Patent Application Publication
(10) Pub. No.: US 2023/0118459 A1
(43) Pub. Date: Apr. 20, 2023

(54) ANTIVIRAL TREATMENT

(71) Applicants: ECOSYNTH NV, Deinze (BE); TRANSMARE CHEMIE NV, Antwerpen (BE)

(72) Inventors: Koen Jeanne Alfons Van Aken, Kuurne (BE); Bart Antoon Judith Ruttens, Wetteren (BE); Jan Karel Emiel Tuur Berton, Sint-Denijs-Westrem (BE); Charlotte Courtens, Drongen (BE); Bram Peter Hilde Bode, Sint-Denijs-Boe (BE)

(21) Appl. No.: 17/909,650

(22) PCT Filed: Mar. 4, 2021
(86) PCT No.: PCT/EP2021/055504

§ 371 Date: Sep. 6, 2022

(30) Foreign Application Priority Data
Mar. 5, 2020 (GB) 2003240.5

(51) Int. Cl. A61K 31/165; A61P 31/12

(52) U.S. Cl. CPC A61K 31/165; A61P 31/12

(57) ABSTRACT

The present description relates to the use of myramistin, its derivatives and the forms thereof for treating or ameliorating infections caused by enveloped viruses, methods for preparing the compounds and pharmaceutical compositions containing such com pounds. In particular the use of myramistin, its derivatives and forms for treating or ameliorating infections caused by coronaviruses. More particularly, the present description relates to the use of myramistin, its derivatives and forms thereof for treating or ameliorating Coronavirus disease 2019 (COVID-19).

### FIELD OF THE INVENTION

The present description relates to the use of myramistin, its derivatives and forms thereof for treating or ameliorating infections caused by enveloped viruses, methods for preparing the compounds and pharmaceutical compositions containing such compounds. In particular the use of myramistin, its derivatives and forms thereof for treating or ameliorating infections caused by respiratory viruses such as RSV, human rhinovirus, influenza virus, adenovirus and coronavirus. More particularly, the present description relates to the use of myramistin, its derivatives and forms thereof for treating or ameliorating Coronavirus disease 2019 (COVID-19).

### BACKGROUND TO THE INVENTION

Viruses are an essential part of the global ecosystem and their existence usually goes unnoticed except when humans are confronted with an (severe) illness or when there is an impact on the food chain caused by these microorganisms.

Today, substantial measures are in place (by vaccination or medication) to control most of the known pathogenic viruses. However, viruses constantly and randomly mutate, whether or not via an additional animal host, and nobody can predict when a new strain of a pathogenic virus will emerge. In addition, the global travelling makes the spreading of such a new virus strain uncontrollable, especially when transmission occurs via air.

The vulnerability of mankind towards unknown viral strains has recently been shown by the outbreak of SARS-CoV-2. Despite some warning signals in the past (SARS-CoV-1, MERS), the world was not prepared and the pandemic resulted in an immense loss of lives (over 2.5 million globally since the outbreak) and global economic disruption and recession which will be felt for many years.

Undoubtedly another pandemic will occur in the future but its timing, the nature of the virus involved and its impact are unpredictable. SARS-CoV-2 affected predominantly elderly people and people with an underlying medical condition, but the scenario that in a next pandemic another part of the population will severely be affected is not unthinkable.

It is a fact that the world is not prepared to efficiently combat a future unknown viral outbreak. Vaccination has been put forward as the most appropriate solution. However, vaccination can never be the “only solution” as still several drawbacks remain. First, it is a reactive solution and although it took only an impressive one year in case of SARS-CoV-2 to deliver the first doses to the patients in response to an outbreak of the previously unknown SARS-CoV-2 virus, it is always lagging behind. Secondly, the high vaccine specificity towards certain strains brings a level of uncertainty of its efficiency towards slightly mutated strains (e.g., UK variant, South African variant, Brazilian variant). Thirdly, transportation (while maintaining the cold chain) and administration of vaccines to a wide population is always an immense logistic operation. In addition, there is still resistance within part of the population towards vaccination, especially towards vaccines relying on newly developed techniques (such as DNA or RNA vaccines) of which the long-term effects are not yet known.

A proactive solution to efficiently combat and control an unknown viral outbreak can be offered by broad-spectrum antiviral medication with a long shelf live. However, only few licensed and efficacious broad-spectrum antivirals exist. Examples include ribavirin, which functions via nebulous effects on both host and virus proteins, and α-IFN, which produces unwanted side effects and remains impractically expensive for widespread use.

To ensure a broad activity spectrum, a common property of the viruses should be targeted. In this respect, the viral envelope is an attractive target for antiviral therapy as it is present in most of the pathogenic viruses. In addition, since the viral membrane nor its properties are changed by mutation of viral genes, no resistance will emerge as opposed to viral protein-targeting therapies.

A viral replication cycle consists of a stage outside the host cells in the form of a virus particle (virion) and a stage inside the infected cell. In the latter stage, disassembly and synthesis of the viral components occurs, followed by reassembly to produce multiple copies which are released extracellularly.

In its extracellular stage, the virus is most vulnerable and easier to control. A compound that modulates the membrane in a way that the virus is no longer able to enter/infect a host cell, stops the replication cycle of the virus, which is eventually removed by the host's immune system.

Although the viral lipid membrane derives from the host cell, it still represents a discrete and susceptible target for antiviral inhibitors.

First, it differs in composition from cellular membranes as virus assembly occurs at membrane subdomains or lipid sorting is involved.

Secondly, it differs in several biochemical properties. Mammalian cells have a biogenic reparative capacity as they can respond to large (10 μm) or small (<0.2 μm) plasma membrane lesions via several rapid (within seconds) repair processes requiring lesion detection, exocytosis of endosomal organelles, and/or self-sealing lipid repair. In addition, host cells constantly metabolize and recycle fatty acids and other membrane components to replenish and repair their plasma membranes. Virions inherently lack the ability to produce/recycle lipids actively and, unlike their host cells, cannot repair damage to or deformation of their membrane.

Thirdly, it differs in biophysical properties as there are many proteins in the mammalian plasma membrane and cytosol responsible for maintaining plasma membrane rigidity and stabilizing membrane curvature. In addition, the plasma membrane in eukaryotic cells is protected and stabilized by an extracellular matrix and cytoskeleton filaments.

Finally, epithelial cells in the host are not monolayers and, in the event of irreparable damage, are replaced by newly generated cells. In contrast, when an viral envelope is disintegrated, the specific virion ceases to exist and virions are not replaced when the infectivity cycle is broken.

The influence of membrane composition, order and fluidity on virus pathogenicity and how to modulate the physicochemical properties of the virus envelope to achieve a desired inhibitory effect, is still a largely unexplored scientific field.

Only a few molecules are described to interact with the viral envelope, but their intrinsic mechanism of action is still not well understood. Antiviral compounds that appear to induce peroxidation of viral phospholipids, are LJ001 and hypericin. Curcumin intercalates in the lipid HCV envelopes decreasing fluidity and inhibiting binding and fusion (EC₅₀ 8.46 μM) (Anggakusuma et al., 2013). Glycyrrhizin also decreases bilayer fluidity (Harada, 2005) and is weakly active against several otherwise unrelated enveloped viruses, including HIV, IAV, VSV (Harada, 2005), and SARS (Cinatl et al., 2003). Also, the antiviral properties of myramistin are described for a wide range of viruses such as Influenza A, Human Papilloma Virus-1 and 2, Human Immunodeficiency Virus, adenoviruses and coronaviruses.

Coronaviruses are enveloped, positive-sense single-strand RNA viruses and have 25 to 32 kb of genome size. As spike proteins are embedded in a membrane, the name “coronavirus” originates from the Latin corona, meaning halo or crown, and refers to its characteristic appearance.

After their first discovery in chickens in 1937, coronaviruses have since been found in various birds and mammals such as bat, cat, dog, cow, pig and mouse. Coronaviruses are divided into four (4) groups (Alpha-, Beta-, Gamma- and Deltacoronaviruses). The Alphacoronavirus and Betacoronavirus groups primarily infect mammals, and Gammacoronavirus and Deltacoronavirus groups are found in birds. It has been known that coronaviruses cause various diseases such as gastrointestinal and respiratory diseases.

The first coronaviruses that infect humans, HCoV-229E and HCoV-0C43, were discovered in the 1960s, followed by HCoV-NL63 (2004) and HCoV-HKU1 (2005), discovered after the severe acute respiratory syndrome (SARS) pandemic. It is known that they generally relate to upper respiratory tract infections, but may cause serious lung diseases in patients with immune deficiencies. It has been reported that coronavirus infection is increased primarily in the winter and early spring seasons, and it has been known that coronaviruses cause a significant percentage of common colds in human adults.

SARS coronavirus (SARS-CoV), causing severe acute respiratory syndrome, was first discovered in 2003. According to a report of the World Health Organization (WHO), there were 8273 patients and 775 deaths (fatality rate: about 10%) all over the world between 2002 and 2004.

In September 2012, a new type of coronavirus (HCoV-EMC) was identified in severe respiratory disease patients showing SARS-like respiratory symptoms such as hyperthermia, cough, dyspnea and the like. Coronavirus (HCoV-EMC) is different from known viruses, and in May 2013, this novel coronavirus was classified by the name of “Middle East respiratory syndrome-coronavirus (MERS-CoV)” by the Coronavirus Study Group of the International Committee on Taxonomy of Viruses. Because the genetic sequence of this virus is similar to that of Pipistrellus bat CoV-HKU5 and HKU4 found in bats, it was assumed that bats are the most probable infection source. However, an article recently published in The Lancet Infectious Diseases reported that all of 50 sera from Omani dromedary camels had protein-specific antibodies against the MERS-CoV spike. Although the virus itself was not found, such study results mean that those camels were infected by the MERS virus or a similar virus at some time, and it is highly likely that camels are a host of the virus. The first identified case of infection by MERS-CoV occurred in Saudi Arabia in September 2012. After that, there were 2494 cases and 858 deaths (fatality rate: 34.4%) officially reported to the WHO by December 2019.

The major clinical symptoms of MERS are those of pneumonia, such as fever (87%), cough (89%), shortness of breath and the like. Vomiting and diarrhea (35%) also occur in some patients. Renal failure has also been reported in patients having lowered immune function, and fatality rate (34.5%) is very high. Because many cases occurred in the Middle East region (Saudi Arabia, Qatar, etc.), it is assumed that this is an infection region, but the precise infection route is still unclear. There was no evidence of the disease widely spreading between humans, but it has been confirmed that transmission can occur when family members or medical personnel are in close contact with patients. It is assumed that the incubation period is 9 to 12 days, but this varies considerably depending on patients. The Middle East Journal of Management (MEJM, Feb. 2008) reported that the incubation period is 1.9 to 14.7 days (average 5.2 days).

In December 2019, there was an outbreak in China of Coronavirus disease 2019 (COVID-19), an infectious disease caused by SARS-CoV-2, a virus closely related to the SARS virus. The virus is spreading on a worldwide scale. It passes from one person to another via respiratory droplets produced from the airways, often during coughing or sneezing. Time from exposure to onset of symptoms is generally between 2 and 14 days. People may have few symptoms or develop fever, cough, and shortness of breath. Cases can progress to pneumonia and multi-organ failure. The fatality rate is estimated at 1 to 3%. The WHO has declared the 2019-20 coronavirus outbreak to be a Public Health Emergency of International Concern. There is a significant uncertainty of how long the outbreak will last and how far it will spread.

To prevent the disease from spreading, hand washing, maintaining distance from people who are coughing, and not touching one's face with unwashed hands are recommended. When an infection is detected, isolation of the patient is recommended and symptoms are managed with supportive care. Both the WHO and Chinese National Health Commission have published detailed treatment recommendations for hospitalized patients with severe acute respiratory infection (SARI) when a SARS-CoV-2 infection is suspected.

The current research activities are focusing on developing either a vaccine or evaluating drugs which are already approved for other antiviral indication. However, optimistic scenarios foresee at least one year for any approval.

Another method for inactivating coronaviruses is the use of solvents, detergents and antiseptics (L.Ya. Zakstelskaya, A.V. Sheboldov Human and animal coronaviruses. M. Medicine, 1977, 221; General and private virology 1982, v2, 316-339; A.I. Korotyaev, SA. Babichev; Medical Microbiology, Virology, and Immunology, Special literature, 1998, 273. Edited by V.M. Zhdanov, S.Ya. Gaydamovich), but these compounds can usually only be used topically. In this respect, the patent WO 2004/108125 describes the use of myramistin as a coronavirus inactivating agent, but in this reference only activity against HCoV-0C43 has been demonstrated.

It is still unclear in which direction therapies are heading but it is obvious that there is an urgent need for developing alternative medicaments for the treatment and prevention of coronavirus diseases.

It is an objective of the present invention to address this pressing need, in providing a drug for treating or ameliorating infections caused by coronaviruses, in particular for treating Coronavirus disease 2019 (COVID-19). It is another objective of this invention to present compounds that target viral envelopes and have an improved activity and selectivity compared to myramistin as the reference compound.

### SUMMARY OF THE INVENTION

In a first aspect the present invention provides a compound of formula (I) or pharmaceutically acceptable form thereof,

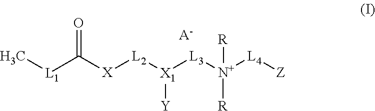

Wherein;
A⁻=pharmaceutically acceptable anion or absent in case Y or Z is an anion-containing group;
X represents O, S or NR₁;
X¹ represents CR₂ or —OP(O)O—; in particular CR₄ more in particular -CH; Y represents hydrogen, OR₂, NR₂, SR₂, SOR₂, SO₂R₂ or an anion-containing group; in particular an anion-containing group selected from the group consisting of oxide (—O—), carboxylate (—COO⁻), sulfonate (—SO₃⁻), sulfate (—OSO₃⁻), phosphate (—OP(O)OR₂)(O⁻), taurinate (—NHC₂H₄SO₃⁻);
Z represents an optionally substituted (hetero)aryl group; in particular an optionally substituted phenyl;
L₁ represents a C₁₀ to C₁₆ aliphatic chain;
L₂, L₃, and L₄ each independently represent C₁₋₆alkyl, O—C₁₋₆eallcyl, S—C₁₋₆alkyl,
SO—C,-alkyl, S02-C1-ealkyl, or NR₃-C1-eallcyl; in particular L₂, L₃, and U each represents C₁₋₆alkyl;
R independently denotes hydrogen or C₁₋₆alkyl; or both R together with the nitrogen atom to which they are attached form a cyclic ring; in particular a 5 or 6-membered ring;
R₁, R₂and R₃ each independently represent hydrogen or C₁₋₆alkyl; for use in the treatment of infections caused by enveloped viruses, with the proviso that the compound of formula (I) is not myristamido-propyl-dimethyl-benzyl-ammonium chloride.

In a second aspect the present invention provides the compounds of formula (I) wherein;
A⁻=pharmaceutically acceptable anion or absent in case Y or Z is an anion-containing group;
X represents O, S or NR₁;
X₁ represents CR₂ or —OP(O)O—; in particular CR₂ more in particular —CH;
Y represents hydrogen or an anion-containing group; in particular oxide (—O⁻),;
Z represents an unsubstituted, monosubstituted or polysubstituted (hetero)aryl group;
in particular an unsubstituted, monosubstituted or polysubstituted substituted phenyl;
L₁ represents a C10-16 alkyl; in particular a C₁₂-alkyl;
L₂, L₃, and L. each independently represent C₁₋₆alkyl, O—C₁₋₆alkyl, S—C₁₋₆alkyl,
SO—C₁₋₆alkyl, SO₂—C₁₋₆alkyl, or NR₃—C₁₋₆alkyl; in particular L₂, L₃, and L. each represents C₁₋₆alkyl;
R independently represents hydrogen or C₁₋₆alkyl;
R₁, R₂and R₃each independently represent hydrogen or C₁₋₆alkyl;
for use in the treatment of infections caused by enveloped viruses, with the proviso that the compound of formula (I) is not myristamido-propyl-dimethyl-benzyl-ammonium chloride.

In a third aspect the present invention provides the compounds of formula (Ia)

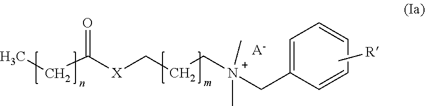

### Wherein;

A⁻=pharmaceutically acceptable anion or absent in case R′ represents an anion containing group;
n is an integer independently selected from 12, 14, or 16; in particular n is 12;
m is an integer from 0 to 5; in particular m is 1;
X represents O, S or NR₁, where R1 represents hydrogen or C₁₋₆alkyl;
R′ represents hydrogen or an anion-containing group selected from the group consisting of carboxylate (—COO⁻), sulfonate (—SO₃₁, sulfate (—OSO₃⁻), phosphate
(—OP(O)(OR₅)(O⁻)), taurinate (—NHC₂H₄SO₃), and oxide (—O⁻), where R₅ is a hydrogen, C₁₋₁₂ alkyl, alkenyl, alkynyl, or aryl;
for use in the treatment of infections caused by enveloped viruses, with the proviso that the compound of formula (I) is not myristamido-propyl-dimethyl-benzyl-ammonium chloride.

In an embodiment according to the invention, it provides the compounds of formula (Ia) wherein;
A⁻=pharmaceutically acceptable anion or absent in case R′ is an anion-containing group;
n is an integer independently selected from 12, 14, or 16; in particular n is 12;
m is an integer from 0 to 5; in particular m is 1;
X represents O or S; in particular X represents O;
R′ represents hydrogen or an anion-containing group selected from the group consisting of carboxylate (—COO⁻), sulfonate (—SO₃⁻) , sulfate (—OSO₃⁻), phosphate (—OP(O)(OR₂)(O⁻) ), taurinate (—NHC₂H₄SO₃⁻), and oxide (—O⁻), where R₂ is a hydrogen, C₁₋₁₂ alkyl, alkenyl, alkynyl, or aryl;
for use in the treatment of infections caused by enveloped viruses.

In an embodiment according to the invention, the compounds of formula (I) are further characterized in one of more of the following elements;
L₁ represents C₁₂alkyl
X represents O, S or NR₁, where R₁ represents hydrogen or C₁₋₆alkyl; in particular R₁ represents hydrogen; more in particular X represents NH;
X represents O or S;
X₁ represents CR₂; in particular —CH;
X₁ represents —OP(O)O— and Y represents oxide (—O⁻); (or expressed differently X₁ and Y taken together form a phosphate group within the chain represented as

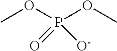

Y represents hydrogen;
L₂ represents CH₂;
L₃ represents CH₂;
L₄ represents C₁₋₄alkyl;
R represents hydrogen or C₁₋₆alkyl; in particular R represents C₁₋₆alkyl; more in particular R represents methyl;
Z represents an optionally substituted (hetero)aryl group selected from the group consisting of phenyl, naphthyl, thionyl, furanyl, pyridinyl, pyrrolyl, oxazolyl, thiazolyl and the like; in particular an optionally substituted phenyl; wherein said (hetero)aryl or phenyl group is optionally substituted with C₁₋₆alkyl, halo, cyano, alkenyl, alkynyl, formyl, carbonyl, alkoxycarbonyl, amido, hydroxy, alkoxy, mercapto, thioalkyl, sulfinyl, sulfonyl, amino, monoalkyl amino, dialkylamino, nitro or an anion-containing group selected from the group consisting of carboxylate (—COO⁻), sulfonate (—SO₃⁻), sulfate (—SO₃⁻), phosphate (—OP(O)(OR₅)(O⁻), taurinate (—NHC₂H₄SO₃⁻), and oxide (—O—), where R₅ is a hydrogen, C₁₋₁₂ alkyl, alkenyl, alkynyl, or aryl; in particular optionally substituted with C₁₋₆alkyl, halo, cyano or an anion-containing group selected from the group consisting of carboxylate (—COO⁻), sulfonate (—SO₃⁻), sulfate (—OSO₃⁻), phosphate (—OP(O)(ORs)(O⁻)), and oxide (—O⁻), where R₅ is a hydrogen; more in particular optionally substituted with C₁₋₆alkyl, halo, cyano or an anion-containing group selected from the group consisting of carboxylate (—COO⁻), sulfonate (—SO₃⁻, and sulfate (—OSO₃⁻); even more in particular optionally substituted with C₁₋₆ alkyl, halo, cyano or an anion-containing group selected from the group consisting of sulfonate (—SO₃⁻, taurinate (—NHC₂H₄SO₃⁻), and sulfate (—OSO₃₁, even more in particular optionally substituted with C₁₋₆alkyl, halo, cyano or an anion-containing group selected from the group consisting of sulfonate (—SO₃⁻, and sulfate (—OSO₃⁻); in a preferred embodiment Z represents a pare substituted phenyl; more in particular a pare substituted phenyl with a substituent selected from C₁₋₆ alkyl, halo, cyano or an anion-containing group selected from the group consisting of sulfonate (—SO₃⁻, and sulfate (—OSO_{31.}

In an embodiment according to the invention, the compounds of formula (Ia) are further characterized in one of more of the following elements;
n is an integer independently selected from 12, or 14; in particular n is 12;
m is 1;
X represents O, S or NR₁, where R₁ represents hydrogen or C₁₋₆alkyl; in particular R₁ represents hydrogen;
X represents O or S;
R′ represents hydrogen or an anion-containing group selected from the group consisting of carboxylate (—COO⁻), sulfonate (—SO₃⁻), sulfate (—OSO₃₁, phosphate (—OP(O)(OR₅)(O⁻), taurinate (—NHC₂H₄SO₃⁻), and oxide (—O—), where R₅ is a hydrogen, C₁₋₁₂ alkyl, alkenyl, alkynyl, or aryl; in particular R′ represents hydrogen or an anion-containing group selected from the group consisting of carboxylate (—COO⁻), sulfonate (—SO₃⁻), sulfate (—OSO₃₁, phosphate (—OP(O)(OR₅)(O⁻), and oxide (—O⁻), where R₅ is a hydrogen; more in particular R′ represents hydrogen or an anion-containing group selected from the group consisting of carboxylate (—COO⁻), sulfonate (—SO₃⁻), and sulfate (—OSO₃⁻); even more in particular R′ represents hydrogen or an anion-containing group selected from the group consisting of sulfonate (—SO₃⁻), taurinate (—NHC₂H₄SO₃⁻), and sulfate (—OSO₃⁻. even more in particular R′ represents hydrogen or an anion-containing group selected from the group consisting of sulfonate (—SO₃⁻), and sulfate (—OSO₃⁻).

In an embodiment according to the invention, the pharmaceutically acceptable anion (A⁻) when present in the compounds of formula (I) or (Ia) is selected from those formed from non-toxic acid addition salts containing pharmaceutically acceptable anions, such as chloride, bromide, sulfate, phosphate, acid phosphate, formate, acetate, salicylate, benzoate, myristate, maleate, fumarate, lactate, lactyl lactate, dodecylsulfate, tartrate, citrate, gluconate, and the like. In one embodiment the pharmaceutically acceptable anion (A⁻) when present in the compounds of formula (I) or (Ia) is selected from those formed from non-toxic acid addition salts containing pharmaceutically acceptable anions, such as sulfate, phosphate, acid phosphate, formate, acetate, salicylate, benzoate, myristate, maleate, fumarate, lactate, lactyl lactate, dodecylsulfate, tartrate, citrate, gluconate, and the like.

In another embodiment the pharmaceutically acceptable anion (A⁻) when present in the compounds of formula (I) or (Ia) is selected from those formed from non-toxic acid addition salts containing pharmaceutically acceptable anions, such as chloride, bromide, sulfate, phosphate, acid phosphate, formate, acetate, citrate, salicylate, benzoate, myristate, lactate, lactyl lactate, dodecylsulfate, and the like; more in particular selected from formate, salicylate, benzoate, myristate, lactyl lactate, dodecylsulfate, and the like. In an even further embodiment the pharmaceutically acceptable anion (A⁻) when present in the compounds of formula (I) or (Ia) is selected from those formed from non-toxic acid addition salts containing pharmaceutically acceptable anions, such as sulfate, phosphate, acid phosphate, formate, acetate, salicylate, benzoate, myristate, lactate, lactyl lactate, dodecylsulfate, and the like; more in particular selected from formate, salicylate, benzoate, myristate, lactyl lactate, dodecylsulfate, and the like.

In a particular embodiment the pharmaceutically acceptable anion (A⁻) in the compounds of formula (I) or (Ia) is represented by the anion of formula (II)

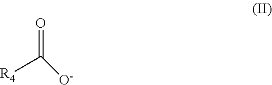

wherein R₄ represents hydrogen, a C₁₋ₗ₃alkyl optionally substituted with one or more substituents independently selected from hydrogen, amino, hydroxyl, aryl, or RB(CO)O—, or R₄ represents an aryl optionally substituted with one or more substituents selected from C₁₋₆ alkyl or hydroxyl; where said R₆ represents hydrogen or a C₁₋₆ alkyl optionally substituted with hydroxyl.

It has been found that the compounds as herein presented are particularly useful in the treatment of infections caused by coronaviruses, such as but not limited to HCoV-229E, HCoV-0C43, HCoV-NL63, HCoV-HKU1, SARS—CoV, MERS—CoV and SARS—CoV-2 (which causes COVID-19). It is accordingly an aspect of the present invention to provide the compounds of formula (I) or (Ia) as herein presented for use in the treatment of infections caused by coronaviruses, such as but not limited to HCoV-229E, HCoV-0C43, HCoV-NL63, HCoV-HKU1, SARS—CoV, MERS-CoV and SARS—CoV-2 (which causes COVID-19); in particular for use in the treatment of infections caused by coronaviruses, such as but not limited to HCoV-HKU1, SARS—CoV, MERS-CoV and SARS—CoV-2 (which causes COVID-19); more in particular for use in the treatment of infections caused by SARS—CoV, and SARS—CoV-2 (which causes COVID-19); even more in particular for use in the treatment of an infection caused by SARS—CoV-2 (which causes COVID-19).

In the treatment of an infection caused by SARS—CoV-2 (which causes COVID-19), the compounds of formula (I) or (la) include myristamido-propyl-dimethyl-benzyl-ammonium chloride, and the aforementioned proviso does not apply.

Thus, in one embodiment the present invention provides a compound of formula (I) or pharmaceutically acceptable form thereof,

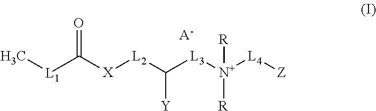

### Wherein;

A⁻=pharmaceutically acceptable anion or absent in case Y or Z is an anion-containing group;

X represents O, S or NR,
Y represents hydrogen, OR₂, NR₂, SR₂, SOR₂, SO₂R₂ or an anion-containing group; in particular an anion-containing group selected from the group consisting of carboxylate
(—COO⁻), sulfonate (—SO₃⁻⁾, sulfate (—OSO₃⁻), phosphate (—OP(O)(OR₂)(O⁻)), taurinate (—NHC₂H₄SO₃);
Z represents an optionally substituted (hetero)aryl group; in particular an optionally substituted phenyl;
L₁ represents a C,o to C,6 aliphatic chain;
L₂, L₃, and L. each independently represents C₁₋₆alkyl, O—C₁₋₆alkyl, S—C₁₋₆alkyl,
SO—C₁₋₆alkyl, SO₂—C₁₋₆alkyl, or NR₃—C₁₋₆alkyl; in particular L₂, L₃, and L₄ each represents C₁₋₆alkyl;
R independently denotes hydrogen or C₁₋₆alkyl;
R₁, R₂ and R₃ each independently represents hydrogen or C₁₋₆alkyl; for use in the treatment of infections caused by SARS—CoV-2 (which causes COVID-19).

In another embodiment the present invention provides a compound of formula (Ia) or pharmaceutically acceptable form thereof,

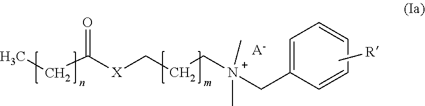

Wherein;
A⁻=pharmaceutically acceptable anion or absent in case R′ is an anion-containing group;
n is an integer independently selected from 12, 14, or 16; in particular n is 12;
m is an integer from 0 to 5; in particular m is 1;
X represents O, S or NR₁, where R₁ represents hydrogen or C₁₋₆ alkyl; in particular R represents O, or NR₁, where R₁ represents hydrogen or C₁₋₆alkyl; even more in particular R represents O, or NR₁, where R₁ represents hydrogen;
R′ represents hydrogen or an anion-containing group; in particular selected from the group consisting of carboxylate (—COO⁻), sulfonate (—SO₃⁻, sulfate (—OSO₃⁻), phosphate (—OP(O)(OR₅)(O⁻), taurinate (—NHC₂H₄SO₃⁻), and oxide (—O—), where R₅ is a hydrogen, C₁₋₁₂ alkyl, alkenyl, alkynyl, or aryl; more in particular selected from the group consisting of carboxylate (—COO⁻), sulfonate (—SO₃⁻, taurinate (—NHC₂H₄SO₃⁻), and sulfate (—OSO₃⁻); even more in particular R′ represents hydrogen or an anion-containing group selected from the group consisting of sulfonate (—SO₃⁻), taurinate (—NHC₂H₄SO₃⁻), and sulfate (—OSO₃⁻); even more in particular R′ represents hydrogen or an anion-containing group selected from the group consisting of sulfonate (SO₃₁, and sulfate (—OSO₃⁻) for use in the treatment of infections caused by SARS—CoV-2 (which causes COVID-19).

In a further objective the present invention provides pharmaceutical compositions comprising the compounds as herein provided.

### DETAILED DESCRIPTION OF THE INVENTION

The present invention is based on the finding that salts of quaternary part of myramistin as well as benzyl group analogues of myramistin as shown in formulas (I) and (la) and derivatives are highly effective in treating or ameliorating infections caused by coronaviruses. It accordingly provides the compounds according to formulas (I) and (la) for use in the treatment of infections caused by enveloped viruses, in particular coronaviruses, with the proviso that the compound of formula (Ia) is not myristamido-propyl-dimethyl-benzyl-ammonium chloride.

As used herein, the compounds of formula (I) or (la) accordingly include the pharmaceutically acceptable salts mixtures containing the cation and pharmaceutically acceptable anions, such as the anions of hydrochloric acid, hydrobromic acid, sulfuric acid, phosphoric acid, acetic acid, maleic acid, fumaric acid, lactic acid, tartaric acid, citric acid, gluconic acid and other described in The Handbook of Pharmaceutical Salts (ISBN 978-3-906390-58-1) in their, if applicable, racemic or stereochemical pure form.

As used herein, the term “form” means a compound of Formula (I) or a compound of Formula (Ia) having a form selected from the group consisting of a free acid, free base, prodrug, salt, hydrate, solvate, clathrate, isotopologue, racemate, enantiomer, diastereomer, stereoisomer, polymorph and tautomer form thereof.

As used herein “myramistin”, is also known as myristamido-propyl-dimethyl-benzyl-ammonium chloride OR (CAS Nr.:15809-19-5) OR (CAS Nr.: 91481-38-8) OR (miramistin) OR (miramystin) OR (einecs 239-908-9) OR (benzyldimethyl(3-tetradecamidopropyl)ammonium chloride) OR (ammonium, benzyldimethyl(3-myristamidopropyl), chloride) OR (benzenemethanaminium, N, N-dimethyl-n-(3-((1-oxotetradecyl)amino)propyl), chloride) OR (benzyldimethyl(3-((1-oxotetradecyl)amino)propyl)ammonium chloride)).

As used herein “a C,o to C,6 aliphatic chain”, is meant to have its generally accepted meaning, and refers to non-aromatic hydrocarbons. Aliphatic compounds can be saturated, joined by single bonds (alkanes), or unsaturated, with one or more double bonds (alkenes), one or more triple bonds (allcynes) or combinations thereof. Besides hydrogen, other elements can be bound to the carbon chain, the most common being oxygen, nitrogen, sulfur, and chlorine.

The compounds of the present description have demonstrated an ability to inhibit the replication of a wide variety of envelope viruses, including respiratory viruses such as RSV, human rhinovirus, influenza virus, adenovirus and coronavirus coronaviruses. The instant compounds possess in vitro activity. In addition to monotherapeutic use, the instant compounds are useful in combination therapy with current standard of antiviral agents, having additive or synergistic activity with one or more known antiviral agents. Besides the use of the compounds as herein provided for use in the treatment of infections caused by coronaviruses, the invention also provides a method of treating or ameliorating a coronaviral infection in a subject in need thereof, comprising administering to the subject an effective amount of a compound of Formula (I) or a form thereof having activity against infections caused by coronaviruses, such as but not limited to HCoV-229E, HCoV-0C₄₃, HCoV-NL63, HCoV-HKU1, SARS—CoV, MERS—CoV and SARS—CoV-2 (which causes COVID-19).

As used herein, the terms “effective amount” or “therapeutically effective amount” mean an amount of compound of Formula (I) or a form, composition or medicament thereof effective in inhibiting the above-noted diseases and thus producing the desired therapeutic, ameliorative, inhibitory or preventative effect in a subject in need thereof. Within the scope of the present description, the “effective amount” of a compound of Formula (I) or a form thereof for use in the manufacture of a medicament, the preparation of a pharmaceutical kit or in a method of treating or ameliorating coronaviral infections; in particular for treating or ameliorating coronaviral infections caused by SARS—CoV, and SARS—CoV-2 (which causes COVID-19); more in particular for treating or ameliorating coronaviral infections caused by SARS—CoV-2 (which causes COVID-19) in a subject in need thereof, is intended to include an amount in a range of from about 0.001 mg to about 3500 mg administered daily; 1.0 mg to about 3500 mg administered daily; 1.0 mg to about 1500 mg administered daily; 1.0 mg to about 1000 mg administered daily; 10.0 mg to about 600 mg administered daily; 0.5 mg to about 2000 mg administered daily; or, an amount in a range of from about 5.0 mg to about 300 mg administered daily.

More in particular, the compositions may be formulated in an aqueous pharmaceutical formulation comprising a therapeutically effective amount of the compounds of the invention and/or one or more physiologically acceptable salt thereof, having a pH within the range of 6.5 7.5. Conveniently the pH of the formulation according to the invention is adjusted on manufacture within the range 6.5-7.5 by means of the use of suitable buffer salts, for example, potassium dihydrogen orthophosphate and disodium hydrogen orthophosphate or citric acid and disodium hydrogen orthophosphate

A further preferred embodiment of the invention is an aqueous formulation for oral or intranasal administration, comprising a therapeutically effective amount of the compounds of the invention and one or more physiologically acceptable salts dissolved in water, together with buffer salts, a preservative and a viscosity enhancing agent. Optionally, and in particular for oral administration the composition may also contain other conventional excipients such as a sweetener, a flavour and/or flavouring aids.

Suitable buffer salts for the oral or intranasal formulation include potassium dihydrogen orthophosphate and disodium hydrogen orthophosphate or citric acid and disodium hydrogen orthophosphate. Examples of suitable viscosity enhancing agents include Xanthan gum, sorbitol, glycerol, sucrose or a cellulose derivative such as carboxymethyl cellulose or an ether thereof such as an alkyl and/or a hydroxyallryl ether of cellulose as for example hydroxypropyl methylcellulose. Suitable preservatives include the alkyl hydroxylbenzoates, such as methyl, ethyl, propyl and/or butyl hydroxybenzoates. Suitable sweeteners include saccharin sodium, sodium cyclamate, sorbitol and sucrose.

It may further be convenient to formulate the compounds in the form of nanoparticles which have a surface modifier adsorbed on the surface thereof in an amount sufficient to maintain an effective average particle size of less than 1000 nm. Suitable surface modifiers can preferably be selected from known organic and inorganic pharmaceutical excipients. Such excipients include various polymers, low molecular weight oligomers, natural products and surfactants. Preferred surface modifiers include non-ionic and anionic surfactants.

Yet another interesting way of formulating the compounds according to the invention involves a pharmaceutical composition whereby the compounds are incorporated in hydrophilic polymers and applying this mixture as a coat film over many small beads, thus yielding a composition with good bioavailability which can conveniently be manufactured and which is suitable for preparing pharmaceutical dosage forms for oral administration. Materials suitable for use as cores in the beads are manifold, provided that said materials are pharmaceutically acceptable and have appropriate dimensions and firmness. Examples of such materials are polymers, inorganic substances, organic substances, and saccharides and derivatives thereof.

This invention also involves a stable suspension aerosol formulation suitable for pressurized delivery which comprises (1) an aqueous solution of the compounds (hereinafter also referred to as the medicament or drug) according to the invention, (2) a suitable propellant, and (3) a stabilizer comprising a water addition. A suitable propellant and stabilizer are those which are suitable for administration by inhalation, the inhalation being used for oral and nasal inhalation therapy. For purposes of the aerosol formulations of this invention, which are intended for inhalation into the lungs, the compounds according to the invention are preferably in the form of a simple aqueous solution of the compounds according to the invention at its natural pH. The particulate medicament or drug is present in the aerosol formulations in a therapeutically effective amount, that is, an amount such that the drug can be administered as an aerosol, such as topically, or via oral or nasal inhalation, and cause its desired therapeutic effect, typically preferred with one dose, or through several doses. The particulate drug is administered as an aerosol from a conventional valve, e.g., a metered dose valve.

A therapeutically effective amount of a particular drug can be selected by those of ordinary skill in the art with due consideration of such factors. Generally, a therapeutically effective amount will be from about 0.001 parts by weight to about 2 parts by weight based on 100 parts by weight of the propellant.

A suitable stabilizer is selected. A suitable stabilizer is a “water addition”. As used herein a “water addition” is an amount of water which (1) is added, either initially with other components of the aerosol formulation, e.g., medicament and propellant, or after the other components, e.g., medicament, propellant, are combined and processed, (2) is in addition to the water which is always present and which develops during processing and/or storage of the aerosol formulation, i.e. “developed” or “nascent” formulation water, and (3) is present in an amount which stabilizes the ordinarily unstable medicinal aerosol formulation having nascent formulation water.

The preparations may be prepared in a manner known per se, which usually involves mixing at least one compound according to the invention with the one or more pharmaceutically acceptable carriers, and, if desired, in combination with other pharmaceutical active compounds, when necessary under aseptic conditions. Reference is again made to U.S. Pat. Nos. 6,372,778, 6,369,086, 6,369,087 and 6,372,733 and the further prior art mentioned above, as well as to the standard handbooks, such as the latest edition of Remington's Pharmaceutical Sciences.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labelled); optionally with one or more leaflets containing product information and/or instructions for use. Generally, such unit dosages will contain between 1 and 1000 mg, and usually between 5 and 500 mg, of the at least one compound of the invention, e.g., about 10, 25, 50, 100, 200, 300 or 400 mg per unit dosage.

In accordance with the method of the present invention, said pharmaceutical composition can be administered separately at different times during the course of therapy or concurrently in divided or single combination forms. The present invention is therefore to be understood as embracing all such regimes of simultaneous or alternating treatment and the term “administering” is to be interpreted accordingly.

The invention will now be illustrated by means of the following synthetic and biological examples, which do not limit the scope of the invention in any way.

### Examples

In the synthesis of Myramistin derivatives three approaches have been applied. In a first approach Myramistin derivatives with varying counterions have been prepared by means of anion exchange departing from the standard Myramistin chloride (Scheme 1 below). In a second approach the base structure of Myramistin has been modified, including conversion into an ester derivative (Scheme 2 below) and conversion into a zwitterionic derivative (Scheme 3 below).

1. Synthesis of the Myramistin Salts

The Myramistin salts were prepared from Myramistin via anion exchange with organic acids (RCOOH) on Amberlyst A26 anion exchange resin (Scheme 1).

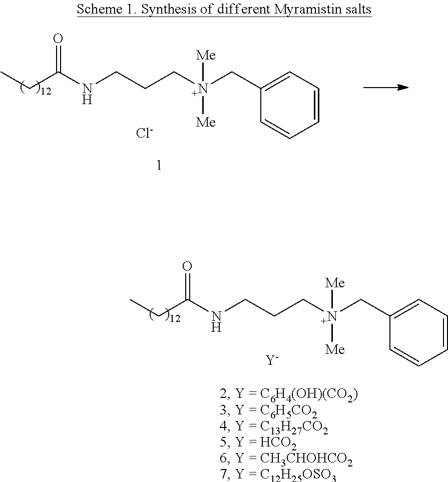

Synthesis of Myramistin salicylate - 2 (ES001387)

Dissolve sodium salicylate (2.552 g, 15.9 mmol) in MeOH (14 mL) and sonicate for 1 min. Add Myramistin chloride (0.925 g, 15.9 mmol) and the solution becomes a suspension. After 2,5 h stirring at room temperature, the solvent was removed under reduced pressure and 50 mL acetone was added. The mixture was sonicated, heated at 45° C. for 5 min and the solids were filtered and were washed with acetone. The filtrate was concentrated in vacuo to give 8.226 g Myramistin salicylate 2 (15.2 mmol, 95% yield). ¹H NMR analysis (CDCl₃): δ8.53 (1H, br t, J=5.2 Hz), 7.96 (1H, dd, J=1.8, 7.7 Hz), 7.56-7.39 (5H, m), 7.26 (1H, dt, J=1.8, 7.2 Hz), 6.86 (1H, dd, J=1.0, 8.2 Hz), 6.76 (1H, dt, J=1.1, 7.6 Hz), 4.57 (2H, s), 3.92 (2H, m), 3.39 (2H, m), 3.02 (6H, s), 2.21 (2H, br t, J=7.8 Hz), 2.11 (2H, m), 1.56 (2H, m), 1.35-1.15 (20H, m), 0.88 (3H, br t, J=6.8 Hz).

Synthesis of Myramistin benzoate - 3 (E5001388)

For the synthesis of Myramistin benzoate, 2.5 g Amberlyst A26 anion exchange resin was conditioned with a solution of benzoic acid in methanol (1.221 g in 20 mL solvent) for a contact time of 30 minutes. Next, the resin was washed with methanol until the pH of the eluent was no longer acidic. Myramistin chloride (0.438 g, 1 mmol) was dissolved in 20 mL MeOH and brought on the resin. The eluent was collected in fractions and the presence of the Myramistin derivative was checked with TLC-UV. The solvent was evaporated under reduced pressure to give 0.515 g Myramistin benzoate 3 (0.98 mmol, 98% yield). ¹H NMR analysis (DMSO-d6): δ 8.25 (1H, br t, J=5.6 Hz), 7.86 (2H, m), 7.58-7.44 (5H, m), 7.39-7.25 (3H, m), 4.53 (2H, s), 3.27 (2H, m), 3.12 (2H, m), 2.95 (6H, s), 2.05 (2H, br t, J=7.5 Hz), 1.94 (2H, m), 1.45 (2H, m), 1.32-1.11 (20H, m), 0.85 (3H, br t, J=6.8 Hz).

Synthesis of Myramistin myristate - 4 (ES001389)

For the synthesis of Myramistin myristate, 2.5 g Amberlyst A26 anion exchange resin was conditioned with a solution of myristic acid in methanol (2.284 g in 20 mL solvent) for a contact time of 30 minutes. Next, the resin was washed with methanol (3×10 mL). pH neutrality could not be checked since the low solubilty of myristic acid in water. Myramistin chloride (0.438 g, 1 mmol) was dissolved in 20 mL MeOH and brought on the resin. The eluent was collected in fractions and the presence of the Myramistin derivative was checked with TLC-UV. The solvent was evaporated under reduced pressure to give 0.563 g Myramistin myristate 4 (0.89 mmol, 89% yield). ¹H NMR analysis (CD30D): δ7.63-7.49 (5H, m), 4.53 (2H, s), 3.36-3.24 (4H, m), 3.04 (6H, s), 2.23-2.01 (6H, m), 1.59 (4H, m), 1.38-1.22 (40H, m), 0.90 (6H, br t, J=6.7 Hz).

Synthesis of Myramistin formate - 5 (E5001390)

For the synthesis of Myramistin formate, 2.5 g Amberlyst A26 anion exchange resin was conditioned with an aqueous solution of formic add (460 mg in 20 mL solvent) for a contact time of 30 minutes. Next, the resin was washed with water until the pH of the eluent was no longer acidic. Myramistin chloride (0.438 g, 1 mmol) was dissolved in 20 mL water and brought on the resin. The eluent was collected in fractions and the presence of the Myramistin derivative was checked with TLC-UV. The mixture was lyophilized yielding 0.148 g of Myramistin formate 5 (0.33 mmol, 33% yield). ¹H NMR analysis (DMSO-d6): δ 8.58 (1 H, s), 8.36 (1 H, br t, J=5.6 Hz), 7.60-7.44 (5H, m), 4.53 (2H, s), 3.27 (2H, m), 3.11 (2H, m), 2.95 (6H, s), 2.05 (2H, t, J=7.5 Hz), 1.94 (2H, m), 1.45 (2H, m), 1.33-1.11 (20H, m), 0.85 (3H, br t, J=6.7 Hz).

Synthesis of Myramistin (L)-lactate - 6 (E5001391)

For the synthesis of Myramistin (L)-lactate, 2.5 g Amberlyst A26 anion exchange resin was conditioned with an aqueous solution of (L)-lactic acid (901 mg, 20 wt % in 20 mL solvent) for a contact time of 30 minutes. Next, the resin was washed with water until the pH of the eluent was no longer acidic. Myramistin chloride (0.438 g, 1 mmol) was dissolved in 20 mL water and brought on the resin. The eluent was collected in fractions and the presence of the Myramistin derivative was checked with TLC-UV. The mixture was lyophilized to give 0.485 g of Myramistin (L)-lactate 6 (0.99 mmol, 99% yield). ¹H NMR analysis (DMSO-d6): δ 8.00 (1H, br t, J=6.0 Hz), 7.59-7.43 (5H, m), 4.51 (2H, s), 3.50 (1H, q, J=6.7 Hz), 3.22 (2H, m), 3.12 (2H, m), 2.94 (6H, s), 2.04 (2H, br t, J=7.5 Hz), 1.93 (2H, m), 1.46 (2H, m), 1.35-1.13 (20H, m), 1.07 (3H, d, J=6.7 Hz), 0.85 (3H, br t, J=6.7 Hz).

Synthesis of Myramistin dodecylsulfate - 7 (ES001392)

Myramistin chloride (500 mg, 1.14 mmol) was dissolved in 5 mL water at room temperature. After 2 minutes of sonication, SDS (sodium dodecylsulfate, 328 mg, 1.14 mmol) was added to the homogenous solution. The SDS seemed to dissolve, while a white solid with a different appearance seemed to precipitate. After 3 hours of stirring at room temperature, the precipitate is collected via filtration. No residual chloride is found and the precipitate is dried under reduced pressure to give Myramystine dodecylsulfate 7 (0.83 mmol, 557 mg). ¹H NMR analysis shows the presence of both anion and cation, in equal proportions. ¹H NMR analysis (DMSO-d6): δ 7.91 (1H, br t, J=5.6 Hz), 7.60-7.43 (5H, m), 4.50 (2H, s), 3.66 (2H, t, J=6.6 Hz), 3.21 (2H, m), 3.11 (2H, m), 2.94 (6H, s), 2.04 (2H, br t, J=7.5 Hz), 1.93 (2H, m), 1.46 (4H, m), 1.38-1.12 (38H, m), 0.85 (6H, br t, J=6.6 Hz).

Synthesis of Myramistin lactateAactyl lactate (2:1) - δ(E5001393)

Since lactic acid inherently tends to dimerize, a Myramistin derivative with a mixture of counterions (which can be roughly be described as 67% lactate, 33% lactyl lactate) has been prepared as well. 2.5 g Amberlyst A26 anion exchange resin was conditioned with an aqueous solution of lactic acid (901 mg) - containing both the monomer and the dimer - for a contact time of 30 minutes. Next, the resin was washed with water until the pH of the eluent was no longer acidic. Myramistin chloride (0.438 g, 1 mmol) was dissolved in 20 mL water and brought on the resin. The eluent was collected in fractions and the presence of the Myramistin derivative was checked with TLC-UV. The mixture was lyophilized to give 0.488 g of Myramistin lactate/lactyl lactate (2:1) δ(0.99 mmol, 99% yield). ¹H NMR analysis (DMSO-d6): δ 7.99 (1H, br t, J=5.7 Hz), 7.59-7.46 (5H, m), 4.70 (0.33H, q, J=7.0 Hz), 4.50 (2H, s), 4.08 (0.33H, q, J=6.8 Hz), 3.61 (1H, q, J=6.7 Hz), 3.21 (2H, m), 3.11 (2H, m), 2.94 (6H, s), 2.04 (2H, br t, J=7.5 Hz), 1.93 (2H, m), 1.45 (2H, m), 1.27 (1H, d, J=7.0 Hz), 1.26 (1H, d, J=6.8 Hz), 1.32-1.14 (20H, m), 1.10 (3H, d, J=6.7 Hz), 0.88 (3H, br t, J=6.7 Hz).

2. Synthesis of the Myramistin Ester Derivative

The Myramistin Ester derivative has been prepared from myristoyl chloride 9 over 3-(Dimethylamino)propyl tetradecanoate 10 according to the following reaction Scheme.

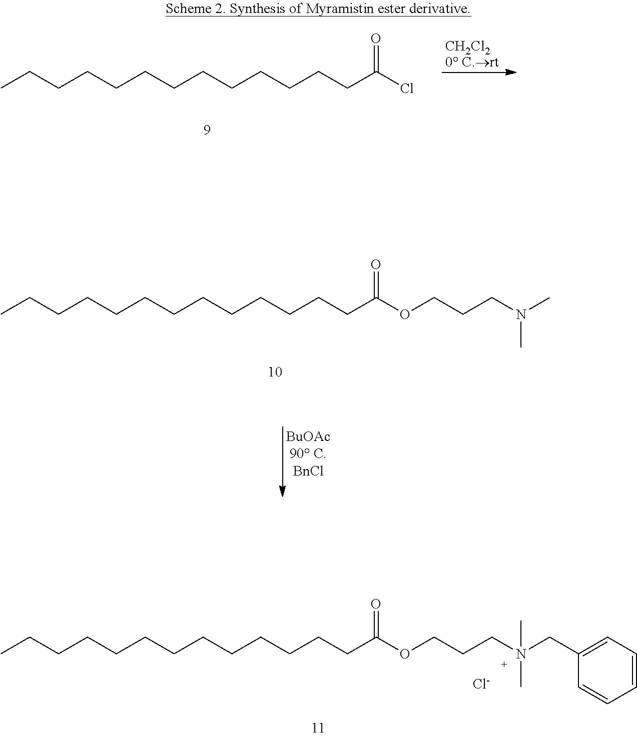

Synthesis of 3-(Dimethylamino)propyl tetradecanoate -10 3-Dimethylamino-1-propanol (3.4 ml, 28.75 mmol) was added to a stirred and cooled (0° C.) solution of myristoyl chloride 9 (6.8 ml, 25 mmol) in dry CH₂Cl₂ (125 ml). The ice bath was removed, and stirring continued under N₂. After 24 h, saturated aqueous NaHCO₃(125 ml) was added and the mixture was stirred vigorously for 45 min. Then, CH₂Cl₂ (125 ml) and H₂O (125 ml) were added and the layers were separated. The organic layer was washed with aqueous NaHCO₃(100 ml) and brine (2×100 ml), dried over MgSO₄, and the solvent was removed under reduced pressure. The resulting oily liquid was filtered (0.45 μm, nylon) and dried under high vacuum. The product 10 (7.246 g) was obtained in 92% yield.

Synthesis of myristamin ester derivative -11 (ES001426)

Benzyl chloride (2.66 ml, 23.11 mmol) was added over 5 min to a stirred solution of 10 (7.245 g, 23.11 mmol) in butyl acetate at 90° C. Stirring was continued at 90° C. under N₂ atmosphere until LC-ELSD showed complete conversion of the starting material (48 h). The mixture was concentrated and the yellow viscous residue was concentrated three times from acetonitrile (3×50 ml). The resulting solid was dissolved in acetonitrile (100 ml) at reflux, allowed to cool to room temperature, and filtered. Then the filtrate was seeded to induce crystallization. The crystals were filtered off, washed with acetonitrile (2×20 ml), and dried to give the target product 11 (6.559 g) in 64% yield. ¹H NMR analysis (CD3OD): δ7.65-7.48 (5H, m), 4.57 (2H, s), 4.20 (2H, t, J=6.0 Hz), 3.42 (2H, m), 3.07 (6H, s), 2.35 (2H, t, J=7.5 Hz), 2.27 (2H, m), 1.61 (2H, m), 1.41-1.22 (20H, m), 0.90 (3H, br t, J=6.7 Hz).

3. Synthesis of the Myramistin Zwitterion Derivative

The zwitterionic derivative of Myramistin has been prepared from the commercially available Myristamide according to the following reaction scheme.

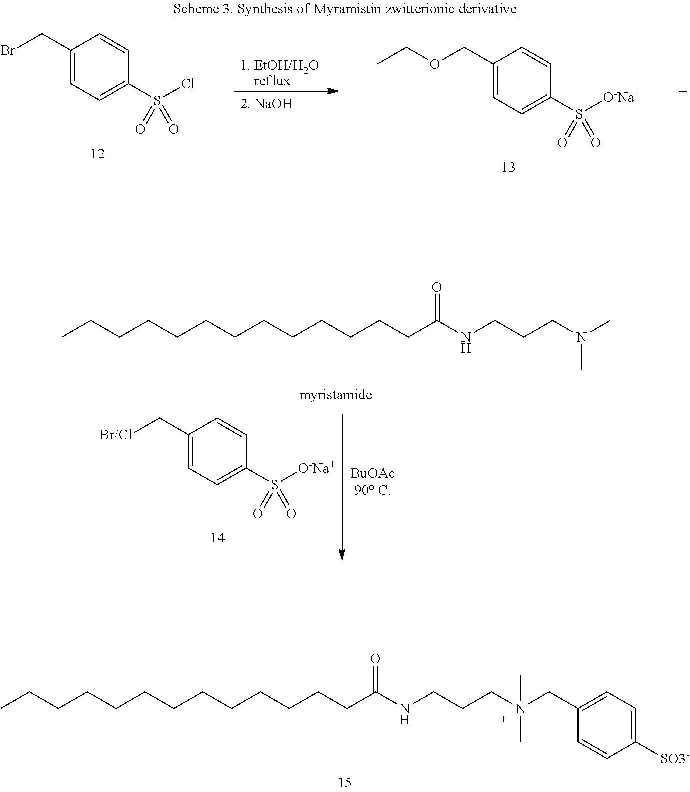

Synthesis of the sodium salt of 4-sulfobenzyl bromide/chloride -14

4-Bromomethylbenzenesulfonyl chloride 12 (95% purity; 2.393 g, 8.43 mmol) in abs. EtOH (5 ml) was heated to reflux, and H₂O (160 pI, 8.85 mmol) was added. The mixture was heated for 3 h, after which it was concentrated. The residue was dissolved in H₂O (7.2 ml), and 2M NaOH (4.2 ml, 8.4 mmol) was added slowly (exothermic). The resulting solution was freeze-dried. ¹H NMR analysis in DMSO-d6 revealed the presence of ethyl ether side product 13 (20-25%), and an aqueous solution of the product was still acidic. Therefore, the product was reconstituted in H₂O (84 ml, 0.1M), and titrated with 0.1M NaOH till a neutral solution was obtained (4.74 ml added). The solution was filtered to remove the turbidity, and the filtrate was freeze-dried. The product (2.078 g) was obtained as an off-white solid. ¹H NMR analysis in DMSO-d6 showed it to be a 60-15/25 mixture of chloride-bromide/ethyl ether, 14/13 respectively. ¹H NMR analysis (DMSO-d6): δ 7.58 (2.6H, m), 7.39 (2H, m), 7.26 (0.7H, m), 4.75 (1.6H, s), 4.70 (0.4H, s), 4.44 (0.6H, s), 3.47 (0.6H, q, J=7.0 Hz), 1.15 (0.9H, t, J=7.0 Hz).

Synthesis of myristamin derivative 3-15 (E5001427)

14/13 (2.04 g) and myristamide ES001387 (2.5 g, δmmol) were heated in butyl acetate at 90° C. under N₂ atmosphere. When LC-UV-ELSD indicated progress of the reaction had stopped (48 h), the mixture was concentrated. The residue was dissolved in MeOH and adsorbed on C₁₈ SiO₂ (20 g). Purification via reversed phase flash chromatography yielded 2.75 g of a product that was further purified by trituration in acetonitrile (110 ml). The solids were filtered off, washed with acetonitrile (2×20 ml) and dried to give the target product 15 (2.524 g) in 83% yield (calculated based on 75% purity of the reagent). ¹H NMR analysis (CD30D): δ7.92 (2H, d, J=8.3 Hz), 7.59 (2H, d, J=8.3 Hz), 4.54 (2H, s), 3.31 (4H, m), 3.03 (6H, s), 2.20 (2H, t, J=7.6 Hz), 2.09 (2H, m), 1.61 (2H, m), 1.41-1.22 (20H, m), 0.90 (3H, br t, J=6.7 Hz).

4. Synthesis of further myramistin salts grouped according to the solubility of the acids required for their preparation.

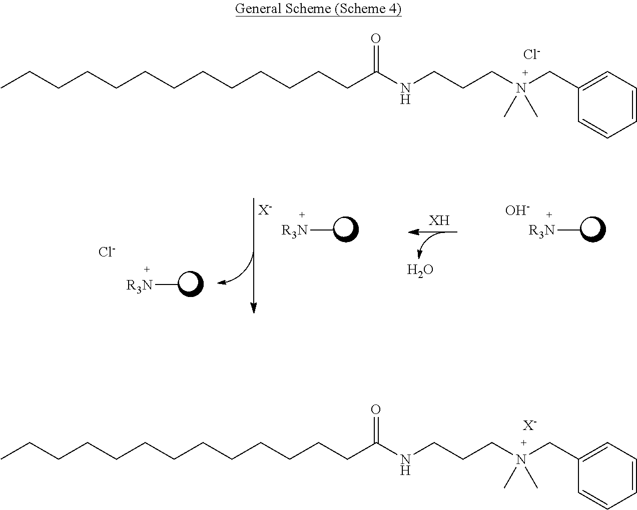

4.a. General procedure for acids soluble in MeOH

Conditioning of the ion exchange resin

An empty SPE cartridge with bottom frit was filled with Amberlyst A26 ion exchange resin, hydroxide form (4 g)*, and a frit was placed on top of the resin. The resin was wetted with MeOH (5 ml). The flowthrough was discarded. A solution of the acid (25 mmol) in MeOH (20 ml) was loaded on top of the ion exchange cartridge and allowed to slowly pass through the column. The flowthrough was discarded. Finally, the ion exchange resin was washed with MeOH (25 ml). The flowthrough was discarded.

Ion exchange - preparation of the Myramistin salt

Myramistin (200 mg, 0.455 mmol) was dissolved in MeOH (1.0 ml) and loaded on top of the conditioned ion exchange column. MeOH (5 ml) was used to elute the product. Both flowthroughs were combined and dried under a stream of N₂. The residue was further dried under high vacuum to yield the Myramistin salt.

*Amberlyst A 26 has >0.8 meq/ml; 4.0 g*0.675 g/ml=5.93 ml resin*0.8 meq/ml=4.74 mmol, i.e., at least 10 eqs. vs. Myramistin; 25 mmol acid corresponds to approximately 5 eqs. vs. the ion exchange resin.

All Myramistin salts thus prepared were analysed by ¹H NMR to confirm structure and purity, and to determine salt ratio. The salt ratio is expressed as quaternary amine/X⁻. For some counterions it is not possible to determine the salt ratio this way.

An overview of the Myramistin salts prepared according to the general procedure for acids soluble in MeOH is given in the Table below.

| | | Compound | FW salt | Yield | Salt |
|---|---|---|---|---|---|
| XH | Acid name | Code | (g/mol) | (%) | ratio |
| 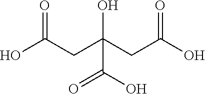 | Citric acid | ES001637 | 594.78 | 82.6 | 1:1 |
| 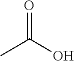 | Acetic acid | ES001643 | 462.71 | 87.4 | 1:1 |
| 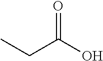 | Propionic acid | ES001642 | 476.74 | 62.0 | 1:1 |
| 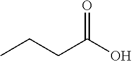 | Butyric acid | ES001641 | 490.77 | 86.3 | 1:1 |
| 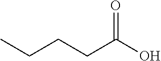 | Valeric acid | ES001644 | 504.79 | 78.4 | 1:1 |
| 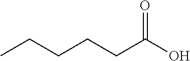 | Caproic acid | ES001640 | 518.82 | 76.4 | 1:1 |
| 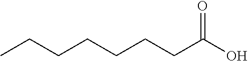 | Caprylic acid | ES001639 | 546.87 | 83.9 | 1:1 |
| 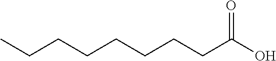 | Pelargonic acid | ES001650 | 560.90 | 89.2 | 1:1 |
| 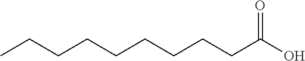 | Capric acid | ES001638 | 574.92 | 77.2 | 1:1 |
| 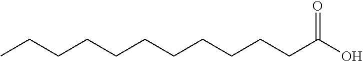 | Lauric acid | ES001633 | 602.98 | 88.3 | 1:1 |
| 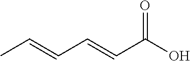 | Sorbic acid | ES001651 | 514.79 | 87.2 | 1:1 |
| 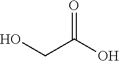 | Glycolic acid | ES001652 | 478.71 | 81.8 | 1:1 |
| 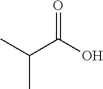 | Isobutyric acid | ES001677 | 490.77 | 61.9 | 1:1 |
| 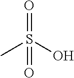 | Methanesulfonic acid | ES001634 | 498.77 | 86.3 | 1:1 |
| 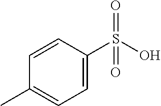 | p-toluenesulfonic acid | ES001635 | 574.87 | 59.2 | 1:1 |
| 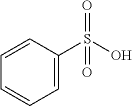 | Benzenesulfonic acid | ES001645 | 560.84 | 72.5 | 1:1 |

4.b.General procedure for acids poorly soluble or Insoluble In MeOH

Conditioning of the ion exchange resin

An empty SPE cartridge with bottom frit was filled with Amberlyst A26 ion exchange resin, hydroxide form (4 g)*, and a frit was placed on top of the resin. The resin was wetted with MeOH (5 ml). The flowthrough was discarded. A solution of the acid (25 mmol) in H₂O (20 ml) was loaded on top of the ion exchange cartridge and allowed to slowly pass through the column. The flowthrough was discarded. Finally, the ion exchange resin was washed with MeOH (25 ml). The flowthrough was discarded.

Ion exchange - preparation of the Myramistin salt

Myramistin (200 mg, 0.455 mmol) was dissolved in MeOH (1.0 ml) and loaded on top of the conditioned ion exchange column. MeOH (5 ml) was used to elute the product. Both flowthroughs were combined and dried under a stream of N₂. The residue was further dried under high vacuum to yield the Myramistin salt.

* Amberlyst A 26 has >0.8 meq/ml; 4.0 g*0.675 g/ml=5.93 ml resin*0.8 meq/ml=4.74 mmol, i.e., at least 10 eqs. vs. Myramistin; 25 mmol acid corresponds to approximately 5 eqs. vs. the ion exchange resin.

All Myramistin salts thus prepared were analysed by ¹H NMR to confirm structure and purity, and to determine salt ratio. The salt ratio is expressed as quaternary amine/X⁻. For some counterions it is not possible to determine the salt ratio this way.

An overview of the Myramistin salts prepared according to the general procedure for acids poorly soluble or insoluble in MeOH is given in the Table below.

| | | Compound | FW salt | Yield | Salt |
|---|---|---|---|---|---|
| XH | Acid name | Code | (g/mol) | (%) | ratio |
| 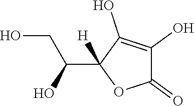 | L-Ascorbic acid | ES001680 | 578.78 | 65.2 | 1:1 |
| 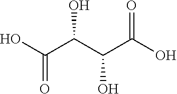 | L-Tartaric acid | ES001700 | 552.75 | 81.7 | 1:0.65 |
| 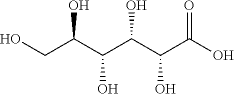 | D-gluconic acid | ES001678 | 598.82 | 84.7 | 1:0.90 |
| 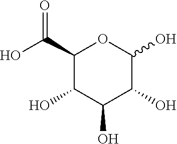 | D-glucuronic aicd | ES001679 | 596.80 | 87.6 | — |
| HI | Hydroiodic acid | ES001673 | 530.57 | 87.2 | — |
| 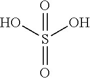 | Sulfuric acid | ES001675 | 500.74 | 84.7 | — |
| 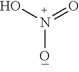 | Nitric acid | ES001676 | 465.67 | 90.5 | — |
| 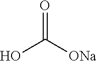 | Sodium bicarbonate | ES001704 | 449.67 | 75.9 | — |
| 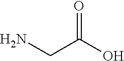 | Glycine | ES001681 | 477.73 | 83.7 | 1:0.70 |
| 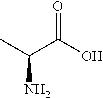 | L-alanine | ES001682 | 491.75 | 92.4 | 1:0.75 |
| 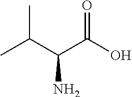 | L-valine | ES001683 | 519.81 | 43.7 | 1:0.90 |
| 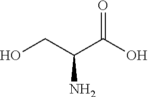 | L-serine | ES001684 | 507.75 | 91.7 | 1:0.9 |
| 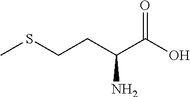 | L-methionine | ES001693 | 551.87 | 76.5 | 1:1 |

4.c. General procedure for acids poorly soluble or Insoluble In H₂O

Conditioning of the ion exchange resin

An empty SPE cartridge with bottom frit was filled with Amberlyst A26 ion exchange resin, hydroxide form (4 g)*, and a frit was placed on top of the resin. The resin was wetted with MeOH (5 ml). The flowthrough was discarded. A solution of the acid (27.5 mmol) in aqueous NaOH (1.25M, 20 ml, 25 mmol) was loaded on top of the ion exchange cartridge and allowed to slowly pass through the column. The flowthrough was discarded. Finally, the ion exchange resin was washed with MeOH (25 ml). The flowthrough was discarded.

Ion exchange - preparation of the Myramistin salt

Myramistin (200 mg, 0.455 mmol) was dissolved in MeOH (1.0 ml) and loaded on top of the conditioned ion exchange column. MeOH (5 ml) was used to elute the product. Both flowthroughs were combined and dried under a stream of N₂. The residue was further dried under high vacuum to yield the Myramistin salt.

* Amberlyst A 26 has >0.8 meq/ml; 4.0 g*0.675 g/ml=5.93 ml resin*0.8 meq/ml=4.74 mmol, i.e., at least 10 eqs. vs. Myramistin; 25 mmol acid corresponds to approximately 5 eqs. vs. the ion exchange resin.

All Myramistin salts thus prepared were analysed by ¹H NMR to confirm structure and purity, and to determine salt ratio. The salt ratio is expressed as quaternary amine/X⁻. For some counterions it is not possible to determine the salt ratio this way.

An overview of the Myramistin salts prepared according to the general procedure for acids poorly soluble or insoluble in H₂O is given in the Table below.

| | | Compound | FW salt | Yield | Salt |
|---|---|---|---|---|---|
| XH | Acid name | Code | (g/mol) | (%) | ratio |
| 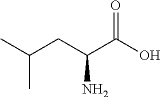 | L-leucine | ES001692 | 533.83 | 92.1 | 1:0.90 |
| 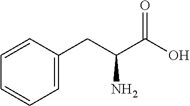 | L-phenylalanine | ES001694 | 567.85 | 89.4 | 1:0.90 |
| 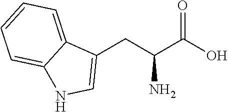 | L-tryptophan | ES001695 | 606.89 | 87.2 | 1:0.95 |
| 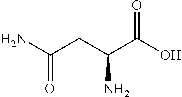 | L-asparagine | ES001696 | 534.78 | 85.6 | 1:0.90 |
| 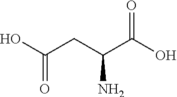 | L-aspartic acid | ES001697 | 535.76 | 87.1 | 1:0.80 |
| 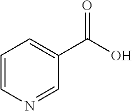 | Nicotinic acid | ES001690 | 525.77 | 86.0 | 1:1 |
| 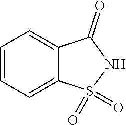 | Saccharin | ES001691 | 585.84 | 85.7 | 1:1 |

4.d.General procedure for Ion exchange with salts of acids

In some cases, it may be beneficial to use the salt of an acid to condition the ion exchange resin.

This approach can be applied when the acid is not commercially available or has poor stability.

Conditioning of the ion exchange resin

An empty SPE cartridge with bottom frit was filled with Amberlyst A26 ion exchange resin, hydroxide form (4 g)*, and a frit was placed on top of the resin. The resin was wetted with MeOH (5 ml). The flowthrough was discarded. A solution of the acid salt (25 mmol) in H₂O (20 ml) was loaded on top of the ion exchange cartridge and allowed to slowly pass through the column. The flowthrough was discarded. Finally, the ion exchange resin was washed with MeOH (25 ml). The flowthrough was discarded.

Ion exchange - preparation of the Myramistin salt

Myramistin (200 mg, 0.455 mmol) was dissolved in MeOH (1.0 ml) and loaded on top of the conditioned ion exchange column. MeOH (5 ml) was used to elute the product. Both flowthroughs were combined and dried under a stream of N₂. The residue was further dried under high vacuum to yield the Myramistin salt.

* Amberlyst A 26 has >0.8 meq/ml; 4.0 g*0.675 g/ml=5.93 ml resin*0.8 meq/ml=4.74 mmol, i.e., at least 10 eqs. vs. Myramistin; 25 mmol acid corresponds to approximately 5 eqs. vs. the ion exchange resin.

All Myramistin salts thus prepared were analysed by ¹H NMR to confirm structure and purity, and to determine salt ratio. The salt ratio is expressed as quaternary amine/X⁻. For some counterions it is not possible to determine the salt ratio this way.

An overview of the Myramistin salts prepared according to the general procedure for ion exchange with salts of acids is given in the Table below.

| | | Compound | FW salt | Yield | Salt |
|---|---|---|---|---|---|
| XH | Acid salt name | Code | (g/mol) | (%) | ratio |
| KBr | Potassium bromide | ES001705 | 483.57 | 87.9 | — |
| KI | Potassium iodide | ES001673 | 568.66 | 75.7 | — |
| NaNO₂ | Sodium nitrite | ES001703 | 465.67 | 39.9 | — |
| 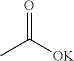 | Potassium acetate | ES001643 | 462.71 | 90.2 | 1:1 |
| 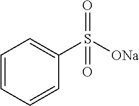 | Sodium benzenesulfonate | ES001645 | 560.84 | 68.4 | 1:1 |
| 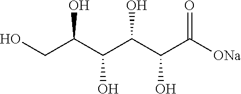 | Sodium gluconate | ES001678 | 598.82 | 72.1 | 1:0.25 |

4.e.¹H NMR data of the Myramistin Salt Derivatives

Myramistin citrate - (ES001637)

¹H NMR (DMSO-d6): δ 7.95 (1H, br t, J=5.8 Hz), 7.48-7.56 (5H, m), 4.51 (2H, s), 3.32 (2H, m), 3.11 (2H, m), 2.94 (6H, s), 2.61 (2H, d, J=15.2 Hz), 2.52 (2H, d, J=15.3 Hz), 2.04 (2H, t, J=7.5 Hz), 1.89-2.06 (2H, m), 1.38-1.49 (2H, m), 1.17-1.28 (20H, m), 0.85 (3H, br t, J=6.8 Hz).

Myramistin caprate - (ES001638)

¹H NMR (DMSO-d6): δ 8.70 (1H, br t, J=5.4 Hz), 7.46-7.57 (5H, m), 4.55 (2H, s), 3.36 (2H, m), 3.11 (2H, m), 2.95 (6H, s), 2.06 (2H, t, J=7.5 Hz), 1.89-1.99 (2H, m), 1.81 (2H, t, J=7.4 Hz), 1.31-1.47 (4H, m), 1.13-1.31 (32H, m), 0.82-0.87 (6H, m).

Myramistin capiylate - (ES001639)

¹H NMR (DMSO-d6): δ 8.63 (1H, br t, J=5.4 Hz), 7.46-7.57 (5H, m), 4.54 (2H, s), 3.32-3.37 (2H, m), 3.13 (2H, m), 2.95 (6H, s), 2.06 (2H, t, J=7.5 Hz), 1.89-1.99 (2H, m), 1.78 (2H, t, J=7.4 Hz), 1.30-1.47 (4H, m), 1.16-1.30 (28H, m), 0.85 (3H, br t, J=6.6 Hz), 0.84 (3H, br t, J=6.8 Hz).

Myramistin laurate - (ES001633)

¹H NMR (DMSO-d6): δ 8.19 (1H, br t, J=5.6 Hz), 7.48-7.55 (5H, m), 4.52 (2H, s), 3.23-3.29 (2H, m), 3.08-3.14 (2H, m), 2.94 (6H, s), 2.05 (2H, t, J=7.5 Hz), 1.86-1.98 (2H, m), 1.84 (2H, t, J=7.4 Hz), 1.30-1.42 (2H, m), 1.42-1.48 (2H, m), 1.15-1.30 (36H, m), 0.83-0.87 (6H, m).

Myramistin caproate - (ES001640)

¹H NMR (DMSO-d6): δ 8.76 (1H, br t, J=5.5 Hz), 7.46-7.58 (5H, m), 4.55 (2H, s), 3.34-3.40 (2H, m), 3.08-3.14 (2H, m), 2.95 (6H, s), 2.06 (2H, t, J=7.5 Hz), 1.81-1.99 (2H, m), 1.78 (2H, t, J=7.4 Hz), 1.34-1.47 (4H, m), 1.15-1.29 (24H, m), 0.80-0.87 (6H, m).

Myramistin butyrate - (ES001641)

¹H NMR (DMSO-d6): δ 8.69 (1H, br t, J=5.5 Hz), 7.46-7.57 (5H, m), 4.55 (2H, s), 3.32-3.38 (2H, m), 3.08-3.14 (2H, m), 2.95 (6H, s), 2.06 (2H, t, J=7.5 Hz), 1.90-1.99 (2H, m), 1.78 (2H, t, J=7.3 Hz), 1.38-1.45 (2H, m), 1.39 (2H, sextet, J=7.3 Hz), 1.18-1.29 (20H, m), 0.85 (3H, br t, J=6.7 Hz), 0.79 (3H, t, J=7.4 Hz).

Myramistin propionate - (ES001642)

¹H NMR (DMSO-d6): δ 8.60 (1H, br t, J=5.5 Hz), 7.46-7.57 (5H, m), 4.54 (2H, s), 3.30-3.36 (2H, m), 3.08-3.14 (2H, m), 2.95 (6H, s), 2.06 (2H, t, J=7.5 Hz), 1.89-2.01 (2H, m), 1.79 (2H, q, J=7.6 Hz), 1.40-1.50 (2H, m), 1.16-1.29 (20H, m), 0.86 (3H, t, J=7.6 Hz), 0.85 (3H, br t, J=6.6 Hz).

Myramistin acetate - (ES001643)

¹H NMR (DMSO-d6): δ 8.64 (1H, br t, J=5.5 Hz), 7.46-7.57 (5H, m), 4.54 (2H, s), 3.30-3.35 (2H, m), 3.08-3.14 (2H, m), 2.95 (6H, s), 2.06 (2H, t, J=7.5 Hz), 1.89-1.99 (2H, m), 1.56 (3H, s), 1.40-1.49 (2H, m), 1.16-1.29 (20H, m), 0.85 (3H, br t, J=6.7 Hz).

Myramistin valerate - (ES001644)

¹H NMR (DMSO-d6): δ 8.22 (1H, br t, J=5.5 Hz), 7.48-7.55 (5H, m), 4.51 (2H, s), 3.23-3.28 (2H, m), 3.09-3.14 (2H, m), 2.94 (6H, s), 2.05 (2H, t, J=7.5 Hz), 1.89-1.97 (2H, m), 1.83 (2H, t, J=7.5 Hz), 1.39-1.49 (2H, m), 1.34-1.39 (2H, m), 1.16-1.28 (22H, m), 0.85 (3H, br t, J=6.8 Hz), 0.82 (3H, t, J=7.3 Hz).

Myramistin pelargonate - (ES001650)

¹H NMR (DMSO-d6): δ 8.40 (1H, br t, J=5.5 Hz), 7.46-7.57 (5H, m), 4.53 (2H, s), 3.27-3.33 (2H, m), 3.08-3.14 (2H, m), 2.94 (6H, s), 2.05 (2H, t, J=7.5 Hz), 1.89-1.98 (2H, m), 1.79 (2H, t, J=7.4 Hz), 1.32-1.50 (4H, m), 1.13-1.29 (30H, m), 0.85 (6H, m).

Myramistin sorbate - (ES001651)

¹H NMR (DMSO-d6): δ 8.46 (1H, t, J=5.5 Hz), 7.46-7.56 (5H, m), 6.61 (1H, dd, J=15.2, 10.9 Hz), 6.02-6.11 (1H, m), 5.75 (1H, qd, J=6.8, 14.6 Hz), 5.63 (1H, dd, J=15.2, 0.5 Hz), 4.54 (2H, s), 3.28-3.33 (2H, m), 3.08-3.14 (2H, m), 2.95 (6H, s), 2.05 (2H, t, J=7.5 Hz), 1.89-1.98 (2H, m), 1.72 (3H, dd, J=6.7, 1.2 Hz), 1.40-1.50 (2H, m), 1.13-1.29 (20H, m), 0.85 (3H, t, J=6.7 Hz).

Myramistin glycolate - (ES001652)

¹H NMR (DMSO-d6): δ 8.06 (1H, br t, J=5.7 Hz), 7.47-7.57 (5H, m), 4.51 (2H, s), 3.58 (2H, s), 3.20-3.25 (2H, m), 3.08-3.15 (2H, m), 2.94 (6H, s), 2.04 (2H, t, J=7.5 Hz), 1.88-1.98 (2H, m), 1.41-1.50 (2H, m), 1.16-1.30 (20H, m), 0.85 (3H, br t, J=6.7 Hz).

Myramistin oxalate - (ES001654)

¹H NMR (DMSO-d6): δ 7.94 (1H, br t, J=5.7 Hz), 7.48-7.55 (5H, m), 4.50 (2H, s), 3.16-3.23 (2H, m), 3.08-3.14 (2H, m), 2.94 (6H, s), 2.04 (2H, t, J=7.5 Hz), 1.88-1.98 (2H, m), 1.41-1.51 (2H, m), 1.17-1.30 (20H, m), 0.85 (3H, br t, J=6.7 Hz).

Myramistin glutarate - (ES001655)

¹H NMR (DMSO-d6): δ 7.96 (1H, br t, J=5.7 Hz), 7.48-7.57 (5H, m), 4.50 (2H, s), 3.18-3.23 (2H, m), 3.08-3.14 (2H, m), 2.94 (6H, s), 2.18 (7H, t, J=7.0 Hz), 2.04 (2H, t, J=7.7 Hz), 1.88 1.97 (2H, m), 1.67 (3.5H, p, J=7.0 Hz), 1.42-1.51 (2H, m), 1.16-1.29 (20H, m), 0.85 (3H, br t, J =6.8 Hz).

Myramistin malonate - (ES001656)

¹H NMR (DMSO-d6): δ 7.91 (1H, br t, J=5.8 Hz), 7.48-7.57 (5H, m), 4.50 (2H, s), 3.17-3.23 (2H, m), 3.08-3.14 (2H, m), 2.94 (6H, s), 2.87 (3.2H, s), 2.04 (2H, t, J=7.5 Hz), 1.87-1.97 (2H, m), 1.41-1.51 (2H, m), 1.17-1.30 (20H, m), 0.85 (3H, br t, J=6.7 Hz).

Myramistin L-malate - (ES001657)

¹H NMR (DMSO-d6): δ 7.91 (1H, br t, J=5.7 Hz), 7.48-7.58 (5H, m), 4.50 (2H, s), 3.98 (1.6H, dd, J=8.8, 4.9 Hz), 3.17-3.23 (2H, m), 3.08-3.14 (2H, m), 2.94 (6H, s), 2.50-2.56 (1.6H, m), 2.35 (1.6H, dd, J=15.6, 4.9 Hz), 2.04 (2H, t, J=7.5 Hz), 1.88-1.98 (2H, m), 1.42-1.51 (2H, m), 1.17-1.30 (20H, m), 0.85 (3H, br t, J=6.7 Hz).

Myramistin succinate - (ES001658)

¹H NMR (DMSO-d6): δ 7.93 (1H, br t, J=5.7 Hz), 7.48-7.55 (5H, m), 4.50 (2H, s), 3.17-3.23 (2H, m), 3.08-3.14 (2H, m), 2.94 (6H, s), 2.31 (s, 7.6H), 2.04 (2H, t, J=7.5 Hz), 1.88-1.97 (2H, m), 1.41-1.51 (2H, m), 1.15-1.30 (20H, m), 0.85 (3H, br t, J=6.7 Hz).

Myramistin maleate - (ES001671)

¹H NMR (DMSO-d6): δ 7.91 (1H, br t, J=5.8 Hz), 7.48-7.57 (5H, m), 6.09 (3H, s), 4.50 (2H, s), 3.17-3.23 (2H, m), 3.08-3.14 (2H, m), 2.94 (6H, s), 2.04 (2H, t, J=7.5 Hz), 1.88-1.98 (2H, m), 1.42-1.50 (2H, m), 1.18-1.30 (20H, m), 0.85 (3H, br t, J=6.7 Hz).

Myramistin fumarate - (ES001672)

¹H NMR (DMSO-d6): δ 8.02 (1H, br t, J=5.7 Hz), 7.47-7.57 (5H, m), 6.52 (4.2H, s), 4.51 (2H, s), 3.19-3.25 (2H, m), 3.08-3.14 (2H, m), 2.94 (6H, s), 2.04 (2H, t, J=7.5 Hz), 1.88-1.98 (2H, m), 1.41-1.50 (2H, m), 1.16-1.29 (20H, m), 0.85 (3H, br t, J=6.7 Hz).

Myramistin methanesulfonate - (ES001634)

¹H NMR (DMSO-d6): δ 7.92 (1H, br t, J=5.8 Hz), 7.48-7.56 (5H, m), 4.51 (2H, s), 3.20-3.24 (2H, m), 3.09-3.14 (2H, m), 2.95 (6H, s), 2.31 (3H, s), 2.04 (2H, t, J=7.5 Hz), 1.87-1.97 (2H, m), 1.42-1.50 (2H, m), 1.18-1.29 (20H, m), 0.85 (3H, br t, J=6.9 Hz).

Myramistin iodide - (ES001673)

¹H NMR (DMSO-d6): δ 7.92 (1H, br t, J=5.7 Hz), 7.48-7.57 (5H, m), 4.51 (2H, s), 3.18-3.24 (2H, m), 3.08-3.14 (2H, m), 2.95 (6H, s), 2.04 (2H, t, J=7.5 Hz), 1.87-1.97 (2H, m), 1.42-1.50 (2H, m), 1.16-1.29 (20H, m), 0.85 (3H, br t, J=6.7 Hz).

Myramistin phosphate - (ES001674)

¹H NMR (DMSO-d6): δ 8.13 (1H, br t, J=5.6 Hz), 7.47-7.57 (5H, m), 4.52 (2H, s), 3.21-3.27 (2H, m), 3.08-3.14 (2H, m), 2.95 (6H, s), 2.05 (2H, t, J=7.5 Hz), 1.88-1.98 (2H, m), 1.41-1.50 (2H, m), 1.15-1.30 (20H, m), 0.85 (3H, br t, J=6.7 Hz).

Myramistin sulfate - (ES001675)

¹H NMR (DMSO-d6): δ 8.86 (1H, br t, J=5.1 Hz), 7.59-7.61 (2H, m), 7.43-7.53 (3H, m), 4.64 (2H, s), 3.44-3.49 (2H, m), 3.07-3.13 (2H, m), 2.97 (6H, s), 2.10 (2H, t, J=7.4 Hz), 1.90-2.00 (2H, m), 1.37-1.48 (2H, m), 1.13-1.29 (20H, m), 0.85 (3H, br t, J=6.7 Hz).

Myramistin p-toluenesulfonate - (ES001635)

¹H NMR (DMSO-d6): δ 7.91 (1H, br t, J=5.8 Hz), 7.47-7.56 (7H, m), 7.09-7.12 (2H, m), 4.51 (2H, s), 3.19-3.23 (2H, m), 3.09-3.14 (2H, m), 2.94 (6H, s), 2.28 (3H, s), 2.04 (2H, t, J=7.5 Hz), 1.89-1.97 (2H, m), 1.42-1.49 (2H, m), 1.18-1.30 (20H, m), 0.85 (3H, br t, J=6.9 Hz).

Myramistin nitrate - (ES001676)

¹H NMR (DMSO-d6): δ 7.91 (1H, br t, J=5.7 Hz), 7.47-7.57 (5H, m), 4.50 (2H, s), 3.19-3.23 (2H, m), 3.08-3.14 (2H, m), 2.94 (6H, s), 2.04 (2H, t, J=7.5 Hz), 1.88-1.97 (2H, m), 1.41-1.50 (2H, m), 1.16-1.30 (20H, m), 0.85 (3H, br t, J=6.7 Hz).

Myramistin isobutyrate - (ES001677)

¹H NMR (DMSO-d6): δ 8.30 (1H, br t, J=5.7 Hz), 7.47-7.57 (5H, m), 4.52 (2H, s), 3.25-3.31 (2H, m), 3.08-3.14 (2H, m), 2.94 (6H, s), 2.05 (2H, t, J=7.5 Hz), 1.87-1.99 (2H, m), 1.91 (1H, heptet, J=6.8 Hz), 1.42-1.50 (2H, m), 1.18-1.30 (20H, m), 0.87 (3H, d, J=6.9 Hz), 0.85 (3H, br t, J=6.8 Hz).

Myramistin D-gluconate - (ES001678)

¹H NMR (MeOD-d4): δ7.51-7.60 (5H, m), 4.53 (2H, s), 4.08 (0.9H, dd, J=3.4, 2.3 Hz), 4.03 (0.9H, d, J=3.5 Hz), 3.79 (0.9H, dd, J=11.1, 3.2 Hz), 3.68-3.76 (1.8H, m), 3.61 (0.9H, dd, J=11.1, 5.7 Hz), 3.27-3.30 (2H, m), 3.04 (6H, s), 2.19 (2H, t, J=7.6 Hz), 2.04-2.12 (2H, m), 1.55 1.62 (2H, m), 1.24-1.34 (20H, m), 0.90 (3H, br t, J=6.9 Hz).

Myramistin D-glucuronate - (ES001679)

¹H NMR (DMSO46): δ7.99 (1H, br t, J=5.7 Hz), 7.47-7.57 (5H, m), 6.49 (0.3H, br s), 6.07 (0.15H, d, J=4.3 Hz), 5.74 (0.15H, d, J=6.7 Hz), 4.98-5.03 (0.3H, m), 4.86 (0.15H, t, J=3.9 Hz), 4.65-4.74 (0.9H, m), 4.51 (2H, s), 4.19-4.31 (0.9H, m), 3.86 (0.15H, d, J=0.7 Hz), 3.76 3.84 (0.3H, m), 3.69 (0.15H, d, J=1.2 Hz), 3.58-3.66 (0.5H, m), 3.19-3.24 (2H, m), 3.07-3.14 (2H, m), 2.99-3.04 (1H, s), 2.94 (6H, s), 2.82-2.89 (0.5H, m), 2.04 (2H, t, J=7.5 Hz), 1.88-1.98 (2H, m), 1.41-1.50 (2H, m), 1.15-1.30 (20H, m), 0.85 (3H, br t, J=6.7 Hz).

Myramistin nicotinate - (ES001690)

¹H NMR (DMSO-d6): δ 8.92 (1H, dd, J=2.0, 0.9 Hz), 8.41 (1H, dd, J=5.0, 1.7 Hz), 8.23 (1H, br t, J=5.7 Hz), 8.06 (1H, ddd, J=7.7, 1.9, 1.9 Hz), 7.47-7.57 (5H, m), 7.23 (1H, ddd, J=7.7, 4.8, 0.9 Hz), 4.53 (2H, s), 3.24-3.30 (2H, m), 3.09-3.15 (2H, m), 2.95 (6H, s), 2.05 (2H, t, J=7.5 Hz), 1.90-1.99 (2H, m), 1.40-1.50 (2H, m), 1.15-1.30 (20H, m), 0.85 (3H, br t, J=6.7 Hz).

Myramistin saccharinate - (ES001691)

¹H NMR (DMSO46): δ7.92 (1H, br t, J=5.7 Hz), 7.47-7.66 (9H, m), 4.50 (2H, s), 3.18-3.24 (2H, m), 3.08-3.15 (2H, m), 2.94 (6H, s), 2.04 (2H, t, J=7.5 Hz), 1.88-1.98 (2H, m), 1.41-1.51 (2H, m), 1.15-1.30 (20H, m), 0.85 (3H, br t, J=6.7 Hz).

Myramistin L-ascorbate - (ES001680)

¹H NMR (MeOD-d4): δ7.51-759 (5H, m), 4.57 (1H, d, J=2.6 Hz), 4.53 (2H, s), 3.87 (1H, td, J=6.7, 2.6 Hz), 3.65-3.70 (2H, m), 3.27-3.30 (4H, m), 3.04 (6H, s), 2.19 (2H, t, J=7.6 Hz), 2.04 2.12 (2H, m), 1.55-1.62 (2H, m), 1.25-1.36 (20H, m), 0.90 (3H, br t, J=6.9 Hz).

Myramistin glycinate - (ES001681)

¹H NMR (MeOD-d4): δ7.51-7.60 (5H, m), 4.53 (2H, s), 3.26-3.30 (4H, m), 3.20 (1.4H, s), 3.04 (6H, s), 2.19 (2H, t, J=7.6 Hz), 2.04-2.12 (2H, m), 1.55-1.62 (2H, m), 1.25-1.34 (20H, m), 0.90 (3H, br t, J=6.9 Hz).

Myramistin L-alanate - (ES001682)

¹H NMR (MeOD-d4): δ7.51-7.60 (5H, m), 4.53 (2H, s), 3.26-3.34 (4.75H, m), 3.04 (6H, s), 2.18 (2H, t, J=7.6 Hz), 2.04-2.12 (2H, m), 1.55-1.62 (2H, m), 1.25-1.34 (22.25H, m), 0.90 (3H, br t, J=6.9 Hz).

Myramistin L-valinate - (ES001683)

¹H NMR (MeOD-d4): δ7.51-7.60 (5H, m), 4.53 (2H, s), 3.26-3.30 (4H, m), 3.16 (0.9H, d, J=4.4 Hz), 3.04 (6H, s), 2.18 (2H, t, J=7.6 Hz), 2.04-2.12 (2.9H, m), 1.55-1.62 (2H, m), 1.24-1.34 (20H, m), 1.00 (2.7H, d, J=7.0 Hz), 0.94 (2.7H, d, J=6.9 Hz), 0.90 (3H, br t, J=6.9 Hz).

Myramistin L-Ieucinate - (ES001692)

¹H NMR (MeOD-d4): δ7.52-7.59 (5H, m), 4.53 (2H, s), 3.24-3.30 (4.9H, m), 3.04 (6H, s), 2.18 (2H, t, J=7.6 Hz), 2.04-2.12 (2H, m), 1.70-1.81 (0.9H, m), 1.55-1.62 (2.9H, m), 1.35-1.42 (0.9H, m), 1.24-1.35 (20H, m), 0.92-0.96 (5.4H, m), 0.90 (3H, br t, J=6.9 Hz).

Myramistin L-serinate - (ES001684)

¹H NMR (MeOD-d4): δ7.51-7.60 (5H, m), 4.53 (2H, s), 3.80 (1H, dd, J=10.8, 4.5 Hz), 3.64 (1H, dd, J=10.8, 6.8 Hz), 3.79 (0.9H, dd, J=10.8, 4.5 Hz), 3.63 (0.9H, dd, J=10.8, 6.8 Hz), 3.34 (0.9H, J=6.8, 4.5 Hz), 3.27-3.30 (4H, m), 3.04 (6H, s), 2.19 (2H, t, J=7.6 Hz), 2.04-2.12 (2H, m), 1.54-1.62 (2H, m), 1.24-1.35 (20H, m), 0.90 (3H, br t, J=6.9 Hz).

Myramistin L-methioninate - (ES001693)

¹H NMR (MeOD-d4): δ7.51-7.60 (5H, m), 4.53 (2H, s), 3.36 (1H, dd, J=7.4, 5.2 Hz), 3.26-3.30 (4H, m), 3.04 (6H, s), 2.52-2.64 (2H, m), 2.18 (2H, t, J=7.5 Hz), 2.09 (3H, s), 1.98-2.12 (3H, m), 1.79-1.91 (1H, m), 1.55-1.62 (2H, m), 1.23-1.35 (20H, m), 0.90 (3H, br t, J=6.8 Hz).

Myramistin L-phenylalaninate - (ES001694)

¹H NMR (MeOD-d4): δ7.51-7.59 (5H, m), 7.26-7.30 (3.6H, m), 7.16-7.22 (0.9H, m), 4.52 (2H, s), 3.48 (0.9H, dd, J=8.3, 4.7 Hz), 3.26-3.29 (4H, m), 3.13 (0.9H, dd, J=13.5, 4.7 Hz), 3.03 (6H, s), 2.79 (0.9H, dd, J=13.5, 8.3 Hz), 2.18 (2H, t, J=7.6 Hz), 2.04-2.11 (2H, m), 1.55-1.62 (2H, m), 1.25-1.34 (20H, m), 0.90 (3H, br t, J=6.9 Hz).

Myramistin L-tryptophanate - (ES001695)

¹H NMR (MeOD-d4): δ7.67-7.70 (0.95H, m), 7.50-7.59 (5H, m), 7.31-7.33 (0.95H, m), 7.14 (0.95H, s), 7.05-7.09 (0.95H, m), 6.97-7.01 (0.95H, m), 4.47 (2H, s), 3.58 (0.95H, dd, J=8.5, 4.4 Hz), 3.29-3.35 (0.95H, m), 3.23-3.27 (4H, m), 2.99 (6H, s), 2.93 (0.95H, dd, J=14.4, 8.5 Hz), 2.18 (2H, t, J=7.6 Hz), 2.00-2.08 (2H, m), 1.55-1.62 (2H, m), 1.24-1.34 (20H, m), 0.90 (3H, br t, J=6.9 Hz).

Myramistin L-asparaginate - (ES001696)

¹H NMR (MeOD-d4): δ7.51-7.60 (5H, m), 4.53 (2H, s), 3.57 (0.9H, dd, J=9.3, 3.8 Hz), 3.27 3.30 (4H, m), 3.04 (6H, s), 2.73 (0.9H, dd, J=15.2, 3.8 Hz), 2.36 (0.9H, dd, J=15.2, 9.3 Hz), 2.19 (2H, t, J=7.6 Hz), 2.06-2.12 (2H, m), 1.55-1.62 (2H, m), 1.24-1.34 (20H, m), 0.90 (3H, br t, J=6.9 Hz).

Myramistin L-aspartate - (ES001697)

¹H NMR (MeOD-d4): δ7.51-7.60 (5H, m), 4.53 (2H, s), 3.70 (0.8H, dd, J=10.6, 3.3 Hz), 3.27 3.30 (4H, m), 3.04 (6H, s), 2.84 (0.8H, dd, J=17.0, 3.3 Hz), 2.51 (0.8H, dd, J=17.0, 10.6 Hz), 2.19 (2H, t, J=7.6 Hz), 2.04-2.12 (2H, m), 1.55-1.62 (2H, m), 1.24-1.34 (20H, m), 0.90 (3H, br t, J=6.8 Hz).

Myramistin trifluoroacetate - (ES001699)

¹H NMR (DMSO-d6): δ 7.94 (1H, t, J=5.8 Hz), 7.46-7.58 (5H, m), 4.51 (2H, s), 3.21 (2H, m), 3.11 (2H, br q, J=6.2 Hz), 2.94 (6H, s), 2.04 (2H, t, J=7.5 Hz), 1.93 (2H, m), 1.46 (2H, m), 1.15-1.31 (20H, m), 0.85 (3H, br t, J=6.7 Hz).

Myramistin benzenesulfonate - (ES001645)

¹H NMR (DMSO46): δ7.91 (1H, t, J=5.8 Hz), 7.57-7.64 (2H, m), 7.46-7.57 (5H, m), 7.27-7.35 (3H, m), 4.50 (2H, s), 3.20 (2H, m), 3.11 (2H, br q, J=6.2 Hz), 2.94 (6H, s), 2.04 (2H, t, J=7.5 Hz), 1.93 (2H, m), 1.45 (2H, m), 1.14-1.33 (20H, m), 0.85 (3H, br t, J=6.8 Hz).

Myramistin L-tartarate - (ES001700)

¹H NMR (DMSO-d6): δ 8.07 (1H, t, J=5.7 Hz), 7.46-7.58 (5H, m), 4.51 (2H, s), 3.72 (1.35H, s), 3.23 (2H, m), 3.11 (2H, br q, J=6.2 Hz), 2.94 (6H, s), 2.05 (2H, t, J=7.5 Hz), 1.93 (2H, m), 1.46 (2H, m), 1.16-1.31 (20H, m), 0.85 (3H, br t, J=6.7 Hz).

Myramistin pyruvate - (ES001701)

¹H NMR (DMSO-d6): δ 7.99 (1H, t, J=5.7 Hz), 7.46-7.58 (5H, m), 4.51 (2H, s), 3.21 (2H, m), 3.11 (2H, br q, J=6.2 Hz), 2.94 (6H, s), 2.04 (2H, t, J=7.5 Hz), 2.01 (3H, s), 1.93 (2H, m), 1.46 (2H, m), 1.16-1.31 (20H, m), 0.85 (3H, br t, J=6.7 Hz).

Myramistin nitrite - (ES001703)

¹H NMR (DMSO-d6): δ 7.99 (1H, t, J=5.8 Hz), 7.45-7.59 (5H, m), 4.51 (2H, s), 3.21 (2H, m), 3.11 (2H, br q, J=6.2 Hz), 2.94 (6H, s), 2.05 (2H, t, J=7.5 Hz), 1.93 (2H, m), 1.46 (2H, m), 1.15-1.32 (20H, m), 0.85 (3H, br t, J=6.7 Hz).

Myramistin carbonate - (ES001704)

¹H NMR (DMSO-d6): δ 8.33 (1H, t, J=5.7 Hz), 7.45-7.59 (5H, m), 4.53 (2H, s), 3.27 (2H, m), 3.11 (2H, br q, J=6.1 Hz), 2.95 (6H, s), 2.05 (2H, t, J=7.5 Hz), 1.94 (2H, m), 1.45 (2H, m), 1.15-1.31 (20H, m), 0.85 (3H, br t, J=6.7 Hz).

Myramistin bromide - (ES001705)

¹H NMR (DMSO-d6): δ 7.93 (1H, t, J=5.7 Hz), 7.47-7.58 (5H, m), 4.52 (2H, s), 3.22 (2H, m), 3.11 (2H, br q, J=6.2 Hz), 2.95 (6H, s), 2.05 (2H, t, J=7.5 Hz), 1.93 (2H, m), 1.46 (2H, m), 1.15-1.31 (20H, m), 0.85 (3H, br t, J=6.7 Hz).

Myramistin perchlorate - (ES001730)

¹H NMR (DMSO-d6): δ 7.95 (1H, t, J=5.7 Hz), 7.46-7.58 (5H, m), 4.51 (2H, s), 3.21 (2H, m), 3.11 (2H, br q, J=6.2 Hz), 2.95 (6H, s), 2.04 (2H, t, J=7.5 Hz), 1.93 (2H, m), 1.46 (2H, m), 1.14-1.32 (20H, m), 0.85 (3H, br t, J=6.7 Hz).

5. Synthesis procedures for the preparation of Myramistin benzyl analogues.

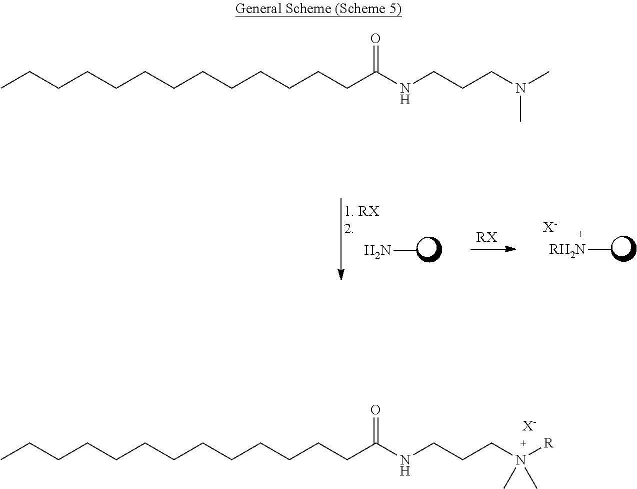

General procedure for quaternization

The starting material (250 mg, 0.8 mmol) was dissolved in ACN (1 ml) by heating to 80° C., after which the reagent (0.84 mmol, 1.05 eq.) was added slowly. Stirring was continued until LC analysis confirmed disappearance of the starting material. Next, tris(2-aminoethyl)amine, polymer-bound (150 mg, 3.5-5.0 mmolg N loading) was added and stirring was continued at 80° C. until complete removal of the reagent was accomplished. The resin was filtered off, washed with MeOH (4 ml) and the combined filtrates were dried under a stream of N₂. The residue was further dried under high vacuum to yield the Myramistin benzyl group analogue.

All Myramistin benzyl group analogues thus prepared were analysed by ¹H NMR to confirm structure and purity.

An overview of the Myramistin benzyl group analogues prepared according to the general procedure for quaternization is given in the Table below.

| | | Compound | | Yield |
|---|---|---|---|---|
| RX | Product | Code | FW (g/mol) | (%) |
| 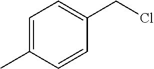 | 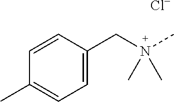 | ES001706 | 453.1 | 57.6 |
| 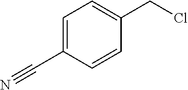 | 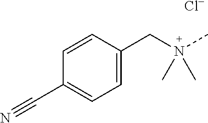 | ES001713 | 464.1 | 57.3 |
| 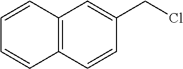 | 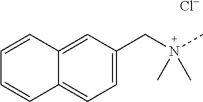 | ES001715 | 489.2 | 56.7 |
| 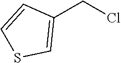 | 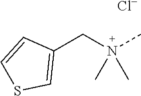 | ES001716 | 445.1 | 54.8 |

Procedure for the preparation of Compound ES001726

Preparation of (2-iodoethyl)benzene

Nal (1.13 g, 7.56 mmol) was added to a stirred solution of (2-chloroethyl)benzene (0.53 g, 4.53 mmol) in acetone (4.2 ml). The mixture was heated to reflux for 3 days, after which it was cooled to room temperature and diluted with Et2O (9 ml). The solids were filtered off. The filtrate was washed with H₂O (2×5 ml), dried over MgSO₄, filtered and concentrated. The crude product was used without further purification.

Quaternization

The starting material (250 mg, 0.8 mmol) was dissolved in ACN (1 ml) by heating to 80° C., after which the reagent (0.84 mmol, 1.05 eq.) was added slowly. Stirring was continued until LC analysis confirmed disappearance of the reagent. Next, the mixture was cooled to room temperature and K₂CO₃ (0.11 g, 0.8 mmol) was added. After 2 h, the solids were removed by filtration, the solvent was removed under reduced pressure and the residue was dried under high vacuum. The residue was reconstituted in ACN (1 ml) and fresh reagent (0.84 mmol, 1.05 eq.) was added slowly at 80° C. Stirring was continued until LC analysis confirmed disappearance of the reagent. Next, the mixture was cooled to room temperature and K₂CO₃ (0.11 g, 0.8 mmol) was added. After 2 h, the solids were removed by filtration, the solvent was removed under reduced pressure and the residue was dried under high vacuum. The residue was reconstituted in ACN (1 ml) and fresh reagent (0.42 mmol, 0.52 eq.) was added slowly at 80° C. Stirring was continued until LC analysis confirmed disappearance of the reagent. The solvent was removed under reduced pressure. The residue was purified by trituration in Et2O to yield the target product.

| RX | Product | Compound Code | FW (g/mol) | Yield (%) |
|---|---|---|---|---|
| 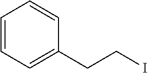 | 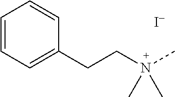 | ES001726 | 544.6 | 31.6 |

¹H NMR data of the Myramistin benzyl analogues

N-(3-(dimethyl 4-methylbenzyl ammonio)propyl)myristamide chloride - (ES001706)

¹H NMR (DMSO-d6): δ 8.07 (1H, t, J=5.7 Hz), 7.43 (2H, d, J=8.0 Hz), 7.30 (2H, d, J=8.0 Hz), 4.49 (2H, s), 3.21 (2H, m), 3.10 (2H, br q, J=6.1 Hz), 2.94 (6H, s), 2.35 (3H, s), 2.05 (2H, t, J=7.5 Hz), 1.92 (2H, m), 1.45 (2H, m), 1.12-1.33 (20H, m), 0.85 (3H, br t, J=6.7 Hz).

N-(3-(dimethyl 4-cyanobenzyl ammonio)propyl)myristamide chloride - (ES001713)

¹H NMR (DMSO-d6): δ 8.03 (1H, t, J=6.0 Hz), 8.00 (2H, d, J=8.2 Hz), 7.77 (2H, d, J=8.2 Hz), 4.64 (2H, s), 3.27 (2H, m), 3.11 (2H, br q, J=6.2 Hz), 2.97 (6H, s), 2.05 (2H, t, J=7.5 Hz), 1.92 (2H, m), 1.45 (2H, m), 1.12-1.33 (20H, m), 0.85 (3H, br t, J=6.7 Hz).

N-(3-(dimethyl 4-methylenenaphthyl ammonio)propyl)myristamide chloride - (ES001715)

¹H NMR (DMSO-d6): δ 8.15 (1H, br s), 7.96-8.12 (4H, m), 7.57-7.68 (3H, m), 4.72 (2H, s), 3.30 (2H, m), 3.13 (2H, br q, J=6.2 Hz), 3.03 (6H, s), 1.92-2.07 (4H, m), 1.41 (2H, m), 1.09-1.31 (20H, m), 0.85 (3H, br t, J=6.7 Hz).

N-(3-(dimethyl 3-methylenethiophenyl ammonio)propyl)myristamide chloride - (ES001716)

¹H NMR (DMSO-d6): δ 7.99 (1H, t, J=5.8 Hz), 7.88 (1H, dd, J=1.2 Hz, 2.9 Hz), 7.72 (1H, dd, J=2.9 Hz, 5.0 Hz), 7.26 (1H, dd, J=1.2 Hz, 5.0 Hz), 4.52 (2H, s), 3.17 (2H, m), 3.10 (2H, br q, J=6.2 Hz), 2.95 (6H, s), 2.05 (2H, br t, J=7.5 Hz), 1.91 (2H, m), 1.46 (2H, m), 1.16-1.31 (20H, m), 0.85 (3H, br t, J=6.7 Hz).

N-(3-(dimethyl ethylenephenyl ammonio)propyl)myristamide chloride - (ES001726)

¹H NMR (DMSO-d6): δ 7.90 (1H, t, J=5.7 Hz), 7.24-7.41 (5H, m), 3.48 (2H, m), 3.32 (2H, m), 3.11 (2H, m), 3.09 (6H, s), 3.00 (2H, m), 2.06 (2H, t, J=7.5 Hz), 1.85 (2H, m), 1.48 (2H, m), 1.15-1.32 (20H, m), 0.85 (3H, br t, J=6.7 Hz).

Biological Examples

The biological and antiviral activity of the compounds can be tested in standard antiviral screening assay such as and in particular provided in the PCT publication WO2004/108125 the assays thereof being incorporated herein by reference.

Cytopathic effect assay:

In 96-well plates, Huh7 cells were seeded at an appropriate density and cultured at 37° C. and 5% CO₂ overnight. Next day, serially diluted compounds (δdoses, in duplicate wells) were added, and then virus was added directly after (no incubation time) the compound treatment. Remdesivir was used as a reference compound. The resulting cultures were kept at 33° C. and 5% CO₂ for an additional 7 days until virus infection in the virus control displayed significant cytopathic effect.

Data:

| | | average | average | | |
|---|---|---|---|---|---|
| | | inhibition | inhibition | | |
| Compound | Indicative | (%) at 14 | (%) at 29 | EC₃₀ | EC₅₀ |
| code | nomenclature | μM | μM | (μM) | (μM) |
| ES001071 | Myramistin | 8.7 | 35.9 | 22.7 | |
| ES001391 | L-Lactate salt | 0 | 18.5 | 45.1 | |
| ES001637 | Citrate salt | 19.5 | 36.8 | 21.1 | |
| ES001652 | Glycolate salt | 17.0 | 36.2 | 23.8 | |
| ES001639 | Caprylate salt | 17.9 | 42.1 | 22.3 | |
| ES001706 | 4-Methyl | 23.8 | 34.1 | | |
| ES001721 | n-propyl | 21.4 | 0 | 6.1 | |
| ES001713 | 4-cyano | 38.3 | 79.4 | 11.8 | 16.7 |
| ES001715 | 2-naphthyl | 45.4 | 0 | 10.0 | |
| ES001726 | ethylphenyl | 26.4 | 14.9 | 15.7 | |
| ES001707 | 3-Methyl | 20.3 | 0 | | |
| ES001688 | 4-linker | 12.3 | 11.4 | | |
| ES001716 | Thiophene | 13.3 | 51.8 | 21.1 | 28.1 |

1. A compound of formula (I) or pharmaceutically acceptable form thereof, 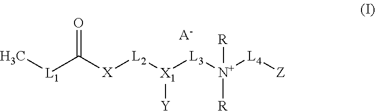
Wherein;
A⁻=pharmaceutically acceptable anion or absent in case Y or Z is an anion-containing group;
X represents O, S or NR,
X₁ represents CR₂ or —OP(O)O—; in particular CR₄ more in particular —CH;
Y represents hydrogen, OR₂, NR₂, SR₂, SOR₂, SO₂R₂ or an anion-containing group; in particular an anion-containing group selected from the group consisting of oxide (—O—), carboxylate (—COO⁻), sulfonate (—SO₃⁻⁾, sulfate (—OSO₃⁻), phosphate (—OP(O)OR₂)(O)), taurinate (—NHC₂H₄SO₃);
Z represents an optionally substituted (hetero)aryl group; in particular an optionally substituted phenyl;
L₁ represents a Cu) to C,6 aliphatic chain;
L₂, L₃, and U each independently represents C₁₋₆alkyl, O—C₁-eallcyl, S—C₁₋₆alkyl,
SO—C,-alkyl, SO₂—C₁-ealkyl, or NR₃—C₁-eallcyl; in particular L₂, L₃, and U each represents C₁₋₆alkyl;
R independently denotes hydrogen or C₁₋₆alkyl; or both R together with the nitrogen atom to which they are attached form a cyclic ring; in particular a 5 or 6-membered ring;
R₁, R₂and R₃ each independently represents hydrogen or C₁₋₆alkyl; for use in the treatment of infections caused by enveloped viruses, with the proviso that the compound of formula (I) is not myristamido-propyl-dimethyl-benzyl-ammonium chloride.

2. The compound for use according to claim 1 wherein;
A⁻=pharmaceutically acceptable anion or absent in case Y or Z is an anion-containing group;
X represents O, S or NR₁;
X₁ represents CR₂ or —OP(O)O—; in particular CR₄ more in particular —CH;
Y represents hydrogen or an anion-containing group; in particular oxide (—O⁻);Z represents an unsubstituted, monosubstituted or polysubstituted (hetero)aryl group; in particular an unsubstituted, monosubstituted or polysubstituted substituted phenyl;
L₁ represents a C₁₀₋₁₆ alkyl; in particular a C₁₂-alkyl;
L₂, L₃, and L. each independently represents C₁₋₆alkyl, O—C₁₋₆alkyl, S—C₁₋₆alkyl,
SO—C₁₋₆alkyl, SO₂—C₁₋₆alkyl, or NR₃—C₁₋₆alkyl; in particular L₂, L₃, and L. each represents C₁₋₆alkyl;
R independently represents hydrogen or C₁₋₆alkyl;
R₁, R₂ and R₃ each independently represents hydrogen or C₁₋₆alkyl; for use in the treatment of infections caused by enveloped viruses, with the proviso that the compound of formula (I) is not myristamido-propyl-dimethyl-benzyl-ammonium chloride.

3. A compound of formula (Ia) or pharmaceutically acceptable form thereof, 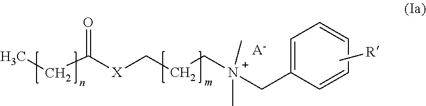
Wherein;
A⁻=pharmaceutically acceptable anion or absent in case R′ represents an anion containing group;
n is an integer independently selected from 12, 14, or 16; in particular n is 12;
m is an integer from 0 to 5; in particular m is 1;
X represents O, S or NR₁, where R₁ represents hydrogen or C₁₋₆alkyl;
R′ represents hydrogen or an anion-containing group selected from the group consisting of carboxylate (—COO⁻), sulfonate (—SO₃), sulfate (—OSO₃⁻), phosphate (—OP(O)(OR₅)(O⁻)), taurinate (—NHC₂H₄SO₃), and oxide (—O⁻), where R₅ is a hydrogen, C₁₋₁₂ alkyl, alkenyl, alkynyl, or aryl;
for use in the treatment of infections caused by enveloped viruses, with the proviso that the compound of formula (I) is not myristamido-propyl-dimethyl-benzyl-ammonium chloride.

4. A compound of formula (Ia) or pharmaceutically acceptable form thereof, 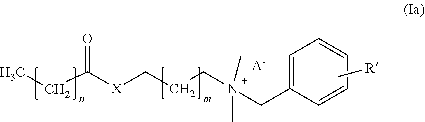
Wherein;
A⁻=pharmaceutically acceptable anion or absent in case R′ is an anion-containing group;
n is an integer independently selected from 12, 14, or 16; in particular n is 12;
m is an integer from 0 to 5; in particular m is 1;
X represents O or S; in particular X represents O;
R′ represents hydrogen or an anion-containing group selected from the group consisting of carboxylate (—COO⁻), sulfonate (—SO₃⁻), sulfate (—OSO₃⁻), phosphate
(—OP(O)(OR₅)(O⁻), taurinate (—NHC₂H₄SO₃⁻), and oxide (—O⁻), where R₅ is a hydrogen, C₁₋₁₂ alkyl, alkenyl, alkynyl, or aryl;
for use in the treatment of infections caused by enveloped viruses.

5. The compounds of claims 1 or 2, wherein one or more of the following restrictions apply;
L₁ represents C₁₂alkyl
X represents O, S or NR₁, where R₁ represents hydrogen or C₁₋₆alkyl; in particular R₁ represents hydrogen; more in particular X represents NH;
X represents O or S;
X₁ represents CR₂; in particular —CH;
X₁ represents —OP(O)O— and Y represents oxide (—O⁻);
Y represents hydrogen;
L₂ represents CH₂;
L₃ represents CH₂;
L₄ represents C₁₋₄alkyl;
R represents hydrogen or C₁₋₆alkyl; in particular R represents C₁₋₆alkyl; more in particular R represents methyl;
Z represents an optionally substituted (hetero)aryl group; in particular an optionally substituted phenyl; wherein said (hetero)aryl or phenyl group is optionally substituted with C₁₋₆alkyl, halo, cyano or an anion-containing group selected from the group consisting of carboxylate (—COO⁻), sulfonate (—SO₃⁻), sulfate (—OSO₃⁻), phosphate (—OP(O)(OR₅)(O⁻)), taurinate (—NHC₂H₄SO₃⁻), and oxide (—O⁻), where R₅ is a hydrogen, C₁₋₁₂ alkyl, alkenyl, alkynyl, or aryl; in particular optionally substituted with C₁₋₆alkyl, halo, cyano or an anion-containing group selected from the group consisting of carboxylate (—COO—), sulfonate (—SO₃⁻), sulfate (—OSO₃₁, phosphate (—OP(OXOR5)(O⁻), and oxide (—O⁻), where R₅ is a hydrogen; more in particular optionally substituted with C,.ealkyl, halo, cyano or an anion-containing group selected from the group consisting of carboxylate (—COO⁻), sulfonate (—SO₃⁻), and sulfate (—OSO₃₁; even more in particular optionally substituted with C₁₋₆alkyl, halo, cyano or an anion-containing group selected from the group consisting of sulfonate (—SO₃⁻), taurinate (—NHC₂H₄SO₃⁻), and sulfate (—OSO₃⁻). even more in particular optionally substituted with C,.ealkyl, halo, cyano or an anion-containing group selected from the group consisting of sulfonate (—SO₃⁻), and sulfate (—OSO₃⁻).

6. The compounds of claims 3 or 4, wherein one or more of the following restrictions apply;
n is an integer independently selected from 12, or 14; in particular n is 12;
m is 1;
X represents O, S or NR₁, where R₁ represents hydrogen or C₁₋₆alkyl; in particular R₁ represents hydrogen;
R′ represents hydrogen or an anion-containing group selected from the group consisting of carboxylate (—COO⁻), sulfonate (—SO₃⁻) , sulfate (—OSO₃₁, phosphate (—OP(O)(OR₅)(O⁻), taurinate (—NHC₂H₄SO₃⁻), and oxide (—O—), where R₅ is a hydrogen, C₁₋₁₂ alkyl, alkenyl, alkynyl, or aryl; in particular R′ represents hydrogen or an anion-containing group selected from the group consisting of carboxylate (—COO⁻), sulfonate (—SO₃ ⁻), sulfate (—OSO₃₁, phosphate (—OP(O)(OR₅)(O⁻), and oxide (—O⁻), where R₅ is a hydrogen; more in particular R′ represents hydrogen or an anion-containing group selected from the group consisting of carboxylate (—COO⁻), sulfonate (—SO₃⁻) , taurinate (—NHC₂H₄SO₃⁻), and sulfate (—OSO₃⁻); even more in particular R′ represents hydrogen or an anion-containing group selected from the group consisting of sulfonate (—SO₃⁻), and sulfate (—OSO₃⁻).

7. The compounds according to any one of claims 1 to 6, wherein when present the pharmaceutically acceptable anion (A⁻) is selected from those formed from non-toxic acid addition salts containing pharmaceutically acceptable anions, such as chloride, bromide, sulfate, phosphate, acid phosphate, formate, acetate, salicylate, benzoate, myristate, lactate, lactyl lactate, dodecylsulfate, and the like; more in particular selected from formate, salicylate, benzoate, myristate, lactyl lactate, dodecylsulfate, and the like.

8. The compounds according to any one of claims 1 to 6, wherein when present the pharmaceutically acceptable anion (A⁻) in the compounds of formula (I) or (la) is represented by the anion of formula (II) 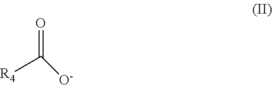
wherein R₄ represents hydrogen, a C₁₋₁₃alkyl optionally substituted with one or more substituents independently selected from hydrogen, amino, hydroxyl, aryl, or R₆(CO)O—, or R₄ represents an aryl optionally substituted with one or more substituents selected from C₁₋₆alkyl or hydroxyl; where said R₆ represents hydrogen or a C,-alkyl optionally substituted with hydroxyl.

9. The compounds according to any one of claims 1 to 8, for use in the treatment of infections caused by coronaviruses, such as but not limited to HCoV-229E, HCoV-0C₄₃, HCoV-NL63, HCoV-HKU1, SARS—CoV, MERS—CoV and SARS—CoV-2; in particular for use in the treatment of infections caused by coronaviruses, such as but not limited to HCoV-HKU1, SARS—CoV, MERS—CoV and SARS—CoV-2; more in particular for use in the treatment of infections caused by SARS—CoV, and SARS—CoV-2; even more in particular for use in the treatment of an infection caused by SARS—CoV-2.

10. A compound of formula (I) or pharmaceutically acceptable form thereof, 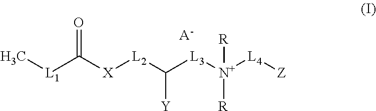
Wherein;
A⁻=pharmaceutically acceptable anion or absent in case Y or Z is an anion-containing group;
X represents O, S or NR₁
Y represents hydrogen, OR₂, NR₂, SR₂, SOR₂, SO₂R₂ or an anion-containing group; in particular an anion-containing group selected from the group consisting of carboxylate (—COO—), sulfonate (SO₃⁻), sulfate (—OSO₃⁻), phosphate (—OP(O)(OR₂)(O⁻) ), taurinate (—NHC₂H₄SO₃”);
Z represents an optionally substituted (hetero)aryl group; in particular an optionally substituted phenyl;
L₁ represents a C₁₀ to C₁₆ aliphatic chain;
L₂, L₃, and L. each independently represents C₁₋₆alkyl, O—C₁₋₆alkyl, S—C₁₋₆alkyl, SO—C,-alkyl, SO₂—C₁₋₆alkyl, or NR₃—C₁₋₆alkyl; in particular L₂, L₃, and L. each represents C₁₋₆alkyl;
R independently denotes hydrogen or C₁₋₆alkyl;
R₁, R₂ and R₃ each independently represents hydrogen or C₁₋₆alkyl; for use in the treatment of infections caused by SARS—CoV-2.

11. A pharmaceutical composition comprising the compounds as defined in any one of claims 1 to 8 or 10.

12. A pharmaceutical composition according to claim 11, consisting of an aqueous pharmaceutical formulation comprising a therapeutically effective amount of the compounds as defined in any one of claims 1 to 8 or 10, dissolved in water and/or one or more physiologically acceptable salt thereof, having a pH within the range of 6.5-7.5.

13. A pharmaceutical composition according to claim 11, consisting of a stable suspension aerosol formulation suitable for pressurized delivery which comprises (1) an aqueous solution of the compounds as defined in any one of claims 1 to 8 or 10, (2) a suitable propellant, and (3) a stabilizer comprising a water addition.
